(19) United States
(12) Patent Application Publication
(10) Pub. No.: US 2026/0256943 A1
(43) Pub. Date: Sep. 3, 2026

(54) NANOPARTICLE POLYMER ENCAPSULATION SYSTEM

(71) Applicant: Core Quantum Technologies, Inc., Columbus, OH (US)

(72) Inventors: Kristie Marie Krug, Upper Arlington, OH (US); Frank Peter Uckert, Columbus, OH (US); Mythreyi Unni, Powell, OH (US)

(21) Appl. No.: 19/414,763

(22) Filed: Dec. 10, 2025

Related U.S. Application Data
(63) Continuation-in-part of application No. 19/251,037, filed on Jun. 26, 2025.
Application No. 19/251,037 is a continuation of application No. 17/943,788, filed on Sep. 13, 2022, now Pat. No. 12,370,266.

(51) Int. Cl. A61K 47/69; A61K 47/68; B82Y 15/00; G01N 33/543; G01N 33/569

(52) U.S. Cl. CPC A61K 47/6931; A61K 47/6883; B82Y 15/00; G01N 33/5434; G01N 33/56972

(57) ABSTRACT

Generally, a nanomaterial polymer encapsulation system useful in the production of nanocomposites comprising an inorganic nanoparticle encapsulated in a hydrophobic region of a polymer with the external hydrophilic region of the polymer ensuring water-solubility and affording a functional group which can be utilized for the production of nanocomposite conjugates.

Specifically, particular embodiments can comprise nanocomposites including one or more inorganic nanoparticles including, metal and non-metal isotopes, a superparamagnetic iron oxide nanoparticle (“SPION”), and/or quantum dots encapsulated in a polystyrene-b-polyethylene glycol affording a functional group that can be activated to conjugate antibodies, modified antibodies, or antibody fragments for the capture of target moieties which in suspension can be nebulized into an inductively coupled plasma time-of-flight mass spectrometry instrument to identify and quantify diverse features of cellular systems.

### I. FIELD OF THE INVENTION

Generally, a nanoparticle polymer encapsulation system useful in the production of nanocomposites comprising an inorganic nanoparticle encapsulated in a hydrophobic region of a polymer with the external hydrophilic region of the polymer ensuring water-solubility and affording a functional group which can be utilized for the production of nanocomposite conjugates.

Specifically, particular embodiments can comprise nanocomposites including one or more inorganic nanoparticles including, metal and non-metal isotopes, superparamagnetic iron oxide nanoparticles, and/or quantum dots encapsulated in a polystyrene-b-polyethylene glycol affording a functional group that can be activated to conjugate antibodies, modified antibodies, or antibody fragments for the capture of target moieties which in suspension can be nebulized into an inductively coupled plasma time-of-flight mass spectrometry instrument to identify and quantify diverse features of cellular systems.

### II. BACKGROUND OF THE INVENTION

Mass cytometry is a powerful tool for high-dimensional and high-throughput single-cell assays. Mass cytometry has become an important tool in the analysis of immune cell function/activation due to its high-parameter capabilities. The application of mass cytometry has significantly expanded since its introduction in 2009. Illustrative examples include immunophenotyping in hematopoietic stem cell transplantation, tumor microenvironment, and cancer immunotherapy.

Until recently, conventional fluorescent-based flow cytometry was the method of choice for phenotypic and functional analysis of single cells. Fluorescent-based flow cytometry measures the total fluorescence in a single sample and then processes the acquired data with an algorithm to mathematically separate the specific fluorophore signals. These data acquisition machines can process up to 50 parameters simultaneously; however, there are practical limitations due to overlapping of emission spectra of fluorescent probes following laser excitation which remains a significant issue in the application of flow cytometry.

Mass cytometry replaces fluorescent labels with non-biologically available metal isotopes with concise mass spectrometry parameters, thereby overcoming the pitfalls associated with overlapping emission spectra, thereby increasing the number of simultaneously analyzable parameters. In mass cytometry, cells are incubated with a mixture of probes/antibodies tagged with a non-radioactive heavy metal isotope. Single-cell suspensions are nebulized such that each droplet contains a single cell. Individual cells subsequently pass through argon plasma, which atomizes and ionizes the single cell contents of the droplet. This converts the cell into a cloud containing ions of the elements present in or on that cell. The low-mass ions are removed from each cloud, resulting in a cloud containing only those ions corresponding to the isotope-conjugated probes. In the time-of-flight chamber, the ions are separated by mass-to-charge ratio. Upon encountering the detector, these ion counts are amplified and converted into electrical signals. Currently, the mass resolution of the detectors allows for multiparametric analysis of up to 130 different parameters.

Another, conventional single-cell technology is single-cell RNA sequencing (scRNAseq), which gives a quantitative measure of gene expression levels per cell. scRNAseq is a powerful genomic tool for dissecting cell populations. However, scRNAseq can only be run on a small number of single cells, whereas mass cytometry experiments can acquire data on several times that number in the range of 10⁶-10⁷ cells, facilitating the characterization of rare cell populations.

However, there remain substantial limitations in the use of mass cytometry for phenotypic and functional analysis of single cells. In this regard, substantial limitations relate to conventional chelating polymers used to selectively bind metal isotopes. In the first instance, it can be a challenge to design a chelating polymer that selectively binds a wide range of different metal isotopes. Hence, in mass cytometry there is a lack of available nanoparticle mass tags. Current versions of mass cytometry can detect 135 channels with a mass range from 75 Da to 209 Da. However, the number of channels used to measure surface or intercellular proteins is currently less than 50 channels due to the lack of available nanoparticle mass tags. While the use of metal-embedding nanocrystals, quantum dots, and polymer dots have been considered promising, their routine usage in mass cytometry for single-cell immunoassays is limited due to nonspecific physical absorption of the nanoparticle-based mass tags into the cell which produces false-positive signals in negative-control cells. Additionally, conventional chelating polymers used for antibody conjugation load a maximum of 100 metal ions, creating a ceiling on signal intensities and making it more difficult to measure extremely weakly expressed markers. Moreover, the large stereoscopic volumes of conventional nanoparticles can result in inefficient diffusion through cell membranes. Further, magnetic beads used to enrich cell populations can interfere with mass cytometry.

Another limitation in the use of convention nanoparticle mass tags in mass cytometry can be that target moiety detection relies on antibody fragment antigen-binding (Fab′-target moiety interaction). Accordingly, the antibody constant fragment (Fc) receptors need to be blocked in order to reduce false positive signal from antibody binding antigen via the Fc region. Where cytotoxic T lymphocytes (CD cells) are target moieties, the additional steps to block the Fc receptors, conventionally include 1) non-specific protein saturation with extra serum or BSA, or 2) staining with anti-CD cell antibodies in a separate first step, prior to the staining with the remainder of the antibody panel.

Accordingly, there would be a substantial advantage in an inventive colloidally stable nanocomposite suitable for use in mass cytometry comprising an encapsulation polymer adapted to: load a wide mass range of inorganic particles to enlarge the number of available mass tags, load a greater number of inorganic particles in the range of about 2 nanometers to about 100 nanometers to enhance signal to noise ratio, load iron oxide to allow magnetic enrichment of cell populations, and form nanocomposites having a hydrodynamic diameter of about 20 nanometers (“nm”) to about 500 nm to increase specific physical adsorption, and in which the antibody binding chemistry does not require conventional steps to block the Fc receptor to reduce nonspecific binding and allows the Fab′-target interaction required for mass cytometry

### III. SUMMARY OF THE INVENTION

A broad object of particular embodiments of the invention can be to provide a nanocomposite comprising one or more nanoparticles encapsulated by a polymer having a hydrophobic region associated with the nanoparticle and a hydrophilic region including a functional group associated with the aqueous environment, wherein the nanoparticle can comprise one or more inorganic particles (“IP”) including, but not necessarily limited to, metal oxide based particles, metal halide based particles, doped metal based particles, non-metal isotopes, metal isotopes (“MI”), quantum dots (“QD”), Perovskite quantum dots, and supraparamagnetic iron oxide nanoparticles (“SPION”), and combinations thereof, wherein the polymer can comprise a polystyrene-b-polyethylene glycol (“PS-b-PEG”) including a functional group wherein polystyrene (“PS”) can have a molecular weight occurring in the range of about 1.5 kDa to about 40 kDa, and wherein the polyethylene glycol (“PEG”) can have a molecular weight occurring in the range of about 10 kDa to about 40 kDa, whereby various combinations and permutations of the IP, the molecular weight of the PS and/or the molecular weight of the PEG, selection of the branched structure of the PEG, and variation in mass ratios thereof, allow for a numerous and wide variety of nanocomposites to be produced having substantially uniform hydrodynamic diameter of 20 nm to 500 nm.

Another broad object of particular embodiments of the invention can be to provide an IP nanocomposite antibody conjugate capable of specifically binding a target moiety. The term “target moiety” as used herein means any molecule found inside an organism, for example, a specific cell, a cell organelle, a protein, a peptide, an amino acid, a nucleic acid, an oligonucleotide, a carbohydrate, a lipid, a metabolite, by selective reaction of a reactive group with the corresponding function groups within the target moiety. Illustrative embodiments of the nanocomposite antibody conjugate include one or more of anti-CD3, anti-CD4, anti-CD8, anti-CD16, or anti-CD32 antibodies capable of specifically binding CD3, CD4, CD 8, CD16 and or CD32 T cells.

Another broad object of particular embodiments of the invention can be to provide methods of isolating IP nanocomposite antibody conjugates bound to a target moiety, wherein SPION nanocomposite antibody conjugates bound to the target moiety can be separated and isolated by influence of a magnetic field, and/or wherein isolated IP nanocomposite antibody conjugates bound to the target moiety can be analyzed and flow sorted into discrete populations based on one or more characteristics of the cells by flow cytometry, and in particular embodiments the isolated IP nanocomposite antibody conjugates bound to the target moiety can be analyzed by mass cytometry.

Naturally, further objects of the invention are disclosed throughout other areas of the specification, drawings, photographs, and claims.

### IV. BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block flow diagram illustrating in general overview the process to make and use elements of the nanoparticle polymer encapsulation system including: polymer synthesis, preparation of polymer encapsulated nanoparticles, antibody preparation, production of polymer encapsulated nanoparticle conjugated antibodies, and polymer encapsulated nanoparticle conjugated antibodies bound to a target moiety.

FIG. 2A is a block flow diagram including Blocks 2A through 2I illustrating a process to synthesize polymers encompassed by the method of FIG. 1, Block 1A including an illustrative example of polystyrene-b-polyethylene glycol amine (“PS-b-PEG-NH₂”).

FIG. 2B is a block flow diagram including Blocks 2A through 2D illustrating an alternate process to synthesize polymers encompassed by the method of FIG. 1, Block 1A including the illustrative example of PS-b-PEG-NH₂.

FIG. 3 is an NMR spectrum which validates the molecular structure of the illustrative example of PS-b-PEG-NH₂ obtained by the polymer synthesis process shown in FIG. 2A, Blocks 2A through 2I or shown in FIG. 2B, Blocks 2A through 2D.

FIG. 4A illustrates an embodiment of a polymer encapsulated nanoparticle (“PENP”) encompassed by FIG. 1, Block 1B including one more inorganic particles (IP) encapsulated in a polymer obtained by the polymer synthesis process shown in FIG. 2A, Blocks 2A through 2I or shown in FIG. 2B, Blocks 2A through 2D (also referred to as an “IsoDot”).

FIG. 4B illustrates an embodiment of polymer encapsulated nanoparticle encompassed by FIG. 1, Block 1B including one more SPION encapsulated in a polymer obtained by the polymer synthesis process shown in FIG. 2A, Blocks 2A through 2I or FIG. 2B, Blocks 2A through 2D (also referred to as a “MagDot”) and which can further encapsulate one or more IP (also referred to as a “MultiDot”).

FIG. 4C illustrates an embodiment of a polymer encapsulated nanoparticle encompassed by FIG. 1, Block 1B wherein the IP comprises one more metal ions (MI) encapsulated in a polymer obtained by the polymer synthesis process shown in FIG. 2A, Blocks 2A through 2I or FIG. 2B, Blocks 2A through 2D.

FIG. 4D illustrates an embodiment of polymer encapsulated nanoparticle encompassed by FIG. 1, Block 1B wherein the IP comprises one more MI and one more SPION encapsulated in a polymer obtained by the polymer synthesis process shown in FIG. 2A, Blocks 2A through 2I or FIG. 2B, Blocks 2A through 2D.

FIG. 4E illustrates an embodiment of polymer encapsulated nanoparticle encompassed by FIG. 1, Block 1B wherein the IP comprises one more QD encapsulated in a polymer obtained by the polymer synthesis process shown in FIG. 2A, Blocks 2A through 2I or FIG. 2B, Blocks 2A through 2D.

FIG. 4F illustrates an embodiment of polymer encapsulated nanoparticle encompassed by FIG. 1, Block 1B including one more QD and one more SPION encapsulated in a polymer obtained by the polymer synthesis process shown in FIG. 2A, Blocks 2A through 2I or FIG. 2B, Blocks 2A through 2D.

FIG. 4G illustrates an embodiment of polymer encapsulated nanoparticle encompassed by FIG. 1, Block 1B including an IP other than a MI and a MI encapsulated in a polymer obtained by the polymer synthesis process shown in FIG. 2A, Blocks 2A through 2I or FIG. 2B, Blocks 2A through 2D.

FIG. 4H illustrates an embodiment of polymer encapsulated nanoparticle encompassed by FIG. 1, Block 1B including one more IP other than an MI, an MI, a QD and a SPION encapsulated in a polymer obtained by the polymer synthesis process shown in FIG. 2A, Blocks 2A through 2I or FIG. 2B, Blocks 2A through 2D.

FIG. 4I, illustrates an embodiment of polymer encapsulated nanoparticle encompassed by FIG. 1, Block 1B including one more IP other than an MI, an MI, a QD or a SPION and combinations thereof encapsulated in a polymer obtained by the polymer synthesis process shown in FIG. 2A, Blocks 2A through 2I or FIG. 2B, Blocks 2A through 2D, wherein the polymer can have a functional group which can be activated to bind one or more agents including as illustrative examples: one or more linkers, a polyethylene glycol, a fluorescent probe, an aptamer, a vitamin, a cell surface receptor, a cell coat, a protein, a peptide, a radioactive isotope, a contrast media, a surface charge modifier, a lectin, or, an antibody, a half antibody, an antibody fragment, and combinations thereof.

FIG. 5 is a block diagram illustrating a method of assembling polymer encapsulated nanoparticles shown in FIGS. 4A through 4H by use of electrohydrodynamic mixing mediated-nanoprecipitation.

FIG. 6 is a micrograph including a 100 nm scale obtained by transmission electron microscopy of a polymer encapsulated nanoparticle encompassed by FIG. 1, Block 1B including MI comprising gold 197 (Au 197) encapsulated in a polymer obtained by the polymer synthesis process shown in FIG. 2A, Blocks 2A through 2I or FIG. 2B, Blocks 2C, 2F, 2H and 2I by use of electrohydrodynamic mixing mediated-nanoprecipitation as illustrated in FIG. 5 (“IsoDot-Au 197”).

FIG. 7A is a micrograph including a 200 nm scale obtained by transmission electron microscopy of a polymer encapsulated nanoparticle encompassed by FIG. 1, Block 1B including IP comprising lead sulfide (“PbS”) encapsulated in a polymer obtained by the polymer synthesis process shown in FIG. 2A, Blocks 2A through 2I or FIG. 2B, Blocks 2C, 2F, 2H and 2I by use of electrohydrodynamic mixing mediated-nanoprecipitation as illustrated in FIG. 5 (“IsoDot-PbS”).

FIG. 7B is a micrograph including a 50 nm scale obtained by transmission electron microscopy of a polymer encapsulated nanoparticle encompassed by FIG. 1, Block 1B including IP comprising PbS encapsulated in a polymer obtained by the polymer synthesis process shown in FIG. 2A, Blocks 2A through 2I or FIG. 2B, Blocks 2C, 2F, 2H and 2I by use of electrohydrodynamic mixing mediated-nanoprecipitation as illustrated in FIG. 5 (“IsoDot-PbS”).

FIG. 8 is a micrograph including a 200 nm scale obtained by transmission electron microscopy of a polymer encapsulated nanoparticle encompassed by FIG. 1, Block 1B comprising IP including MI of indium 113/115 (“In 113,115”) and a non-metal arsenic ion 75 (“As 75”) encapsulated in a polymer obtained by the polymer synthesis process shown in FIG. 2A, Blocks 2A through 2I or FIG. 2B, Blocks 2C, 2F, 2H and 2I by use of electrohydrodynamic mixing mediated-nanoprecipitation as illustrated in FIG. 5 (“IsoDot-In 113, 115/As75”).

FIG. 9A is a micrograph including a 100 nm scale obtained by transmission electron microscopy of a polymer encapsulated nanoparticle encompassed by FIG. 1, Block 1B including MI comprising cadmium 114 (“CD114”) and tellurium 130 (“Te130”) encapsulated in a polymer obtained by the polymer synthesis process shown in FIG. 2A, Blocks 2A through 2I or FIG. 2B, Blocks 2C, 2F, 2H and 2I by use of electrohydrodynamic mixing mediated-nanoprecipitation as illustrated in FIG. 5 (“IsoDot-Cd 114/Te130”).

FIG. 9B is a micrograph including a 100 nm scale obtained by transmission electron microscopy of a polymer encapsulated nanoparticle encompassed by FIG. 1, Block 1B including MI comprising CD114 and Te130 encapsulated in a polymer obtained by the polymer synthesis process shown in FIG. 2A, Blocks 2A through 2I or FIG. 2B, Blocks 2C, 2F, 2H and 2I by use of electrohydrodynamic mixing mediated-nanoprecipitation as illustrated in FIG. 5 (“IsoDot-Cd 114/Te130”).

FIG. 10 is a micrograph including a 100 nm scale obtained by transmission electron microscopy of a polymer encapsulated nanoparticle encompassed by FIG. 1, Block 1B including one or more SPION 20 nm encapsulated in a polymer obtained by the polymer synthesis process shown in FIG. 2A, Blocks 2A through 2I or FIG. 2B, Blocks 2C, 2F, 2H and 2I by use of electrohydrodynamic mixing mediated-nanoprecipitation as illustrated in FIG. 5 (“MagDot₂₀ₙₘ”).

FIG. 11 is a micrograph including a 100 nm scale obtained by transmission electron microscopy of a polymer encapsulated nanoparticle encompassed by FIG. 1, Block 1B including one or more SPION 15 nm encapsulated in a polymer obtained by the polymer synthesis process shown in FIG. 2, Blocks 2A, Blocks 2A through 2I or FIG. 2B, Blocks 2C, 2F, 2H and 2I by use of electrohydrodynamic mixing mediated-nanoprecipitation as illustrated in FIG. 5 (“MagDot₁₅ₙₘ”).

FIG. 12 is a micrograph including a 100 nm scale obtained by transmission electron microscopy of a polymer encapsulated nanoparticle encompassed by FIG. 1, Block 1B including one or more SPION 5 nm encapsulated in a polymer obtained by the polymer synthesis process shown in FIG. 2, Blocks 2A through 2I or FIG. 2B, Blocks 2C, 2F, 2H and 2I by use of electrohydrodynamic mixing mediated-nanoprecipitation as illustrated in FIG. 5 (“MagDot5 nm”).

FIG. 13 is a plot of hydrodynamic diameter against concentration of particles per milliliter of the polymer encapsulated nanoparticles shown in FIG. 9B evidencing a hydrodynamic diameter of about 128 nm±22 nm.

FIG. 14 is a bar graph comparing the iron concentration of polymer encapsulated nanoparticle including a SPION of 15 nm and QD having an emission wavelength occurring at 610 nm to polymer encapsulated nanoparticle including SPION of 5 nm and QD having an emission wavelength occurring at 610 nm.

FIG. 15 is a bar graph comparing the fluorescence of polymer encapsulated nanoparticle including SPION of 15 nm to polymer encapsulated nanoparticle including SPION of 5 nm.

FIG. 16A is a flow cytometry univariant histogram depicting detection of Cyto-Comp human lymphocytes (Cyto-Comp Cell Kit-6607023) with CD45 receptors associated with an IsoDot Au 197 mouse anti-human CD45 (“IsoDot-Au197-Ab-CD45”) and then with a secondary Alexa Fluor® 488 goat anti-mouse IgG, Fab′)₂ encompassed by FIG. 1, Block 1E (“IsoDot-Au197-Alexa Fluor® 488 Ab-Ab Cd45-PBMC CD45”).

FIG. 16B is a flow cytometry univariant histogram depicting detection of Cyto-Comp human lymphocytes (Cyto-Comp Cell Kit-6607023) with CD8 receptors labelled with IsoDot-Cd 106, 114/Se 76 mouse anti-human CD8 (“IsoDot Cd 106,114/Se 76-Ab-CD8-PBMC CD8”) encompassed by FIG. 1, Block 1E.

FIG. 16C is a flow cytometry univariant histogram depicting detection of Cyto-Comp human lymphocytes (Cyto-Comp Cell Kit-6607023) with CD4 receptors labelled with In 113,115/As 75 mouse anti-human CD8 IsoDot (IsoDot In 113,115/As 75-Ab-CD4-PBMC CD4) encompassed by FIG. 1, Block 1E.

FIG. 17A is a flow cytometry bivariant dot plot depicting detection of CD3 positive peripheral blood mononuclear cells bound to mouse anti human CD3 linked to a polymer encapsulated nanoparticle encompassed by FIG. 1, Block 1D including one or more SPION and CD3 negative cells in a reaction solution prior to magnetic separation.

FIG. 17B is a flow cytometry bivariant dot plot depicting detection of depleted CD3 positive peripheral blood mononuclear cells in the non-magnetic fraction of the reaction solution subsequent to magnetic separation.

FIG. 17C is a flow cytometry bivariant dot plot depicting detection of the enriched CD3 positive peripheral blood mononuclear cells bound to mouse anti human CD3 linked to a polymer encapsulated nanoparticle encompassed by FIG. 1, Block 1D including one or more SPION in the magnetic fraction of the reaction solution subsequent to magnetic separation.

FIG. 18 is a mass cytometry dot plot of ion counts per cell representing the signal intensity of CD8 populations identified by their unique heavy metal isotopes in the Cd-106 channel.

FIG. 19 is a mass cytometry dot plot of ion counts per cell representing the signal intensity of CD8 populations identified by their unique heavy metal isotopes in the Cd-114 channel.

FIG. 20 is a mass cytometry dot plot of ion counts per cell representing the signal intensity of CD8 populations identified by their unique heavy metal isotopes in the Te-130 channel.

### V. DETAILED DESCRIPTION OF THE INVENTION

The present invention may be understood by reference to the following detailed description of aspects of the invention and the examples included therein and to the figures and their previous and following description. Compounds, compositions, articles, devices, or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular embodiments for the purpose of enabling a person of ordinary skill in the art to make and use a numerous and wide variety of embodiments of the invention, even if not explicitly disclosed. Unless defined otherwise, all technical and scientific terms used herein have the same meaning as commonly understood by one of ordinary skill in the art to which this invention belongs. Although any methods and materials similar or equivalent to those described herein can be used in the practice of the present invention.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein may be different from the actual publication dates, which may need to be independently confirmed.

As used in the specification and the appended claims, the singular forms “a,” “an” and “the” include plural referents unless the context clearly dictates otherwise. Thus, for example, reference to “a component,” “a polymer,” or “a particle” includes mixtures of two or more such components, polymers, or particles, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, as illustrative examples, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

Disclosed are the components to be used to prepare the compositions of the invention as well as the compositions themselves to be used within the methods disclosed herein. These and other materials are disclosed herein, and it is understood that when combinations, subsets, interactions, and groups of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, elements in methods of making and using the compositions of the invention. Thus, if there are a variety of additional elements that can be performed it is understood that each of these additional elements can be performed with any specific embodiment or combination of embodiments of the methods of the invention. It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### Process Overview.

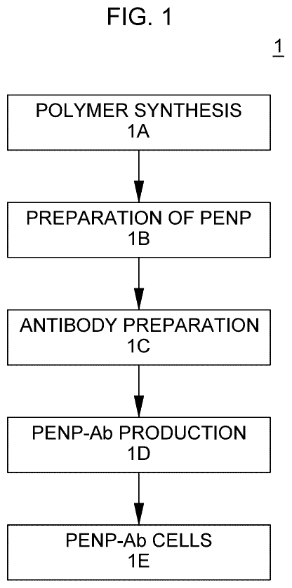

Now, with primary reference to FIG. 1, a block flow diagram provides a general overview of the nanomaterial polymer encapsulation system (1) including one or more of: polymer synthesis of a nanoparticle (“NP”) (2) encapsulation polymer (“P”) (3) (Block 1A); formation of polymer encapsulated nanoparticles (“PENP”) (4) (Block 1B); preparation of antibodies, half-antibodies, antibody fragments (individually or collectively “Ab”) (5) (Block 1C); preparation of PENP antibody conjugates (“PENP-Ab”) (6) (Block 1D); PENP-Ab cell labeling (PENP-Ab-Cells”) (7) (Block 1E).

### Polymer Synthesis.

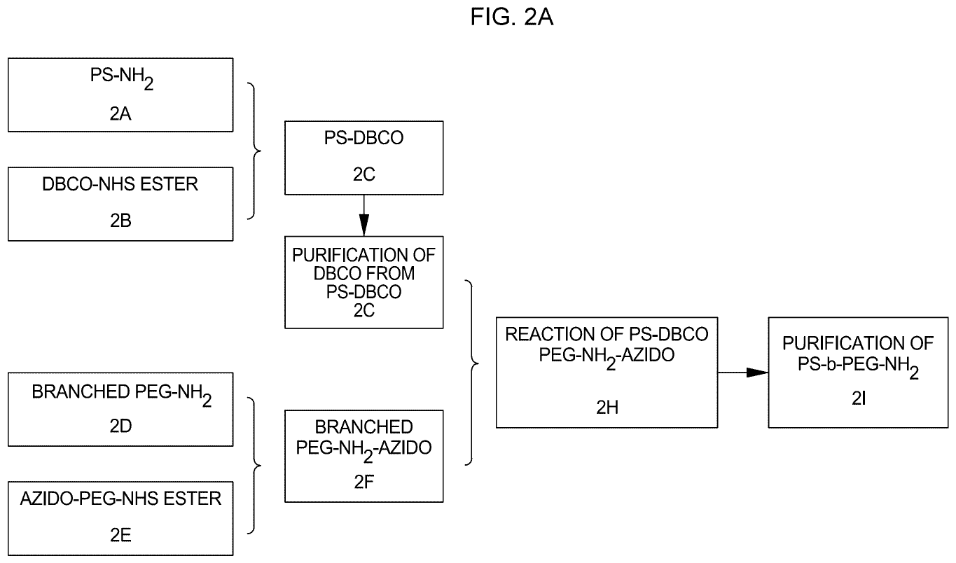

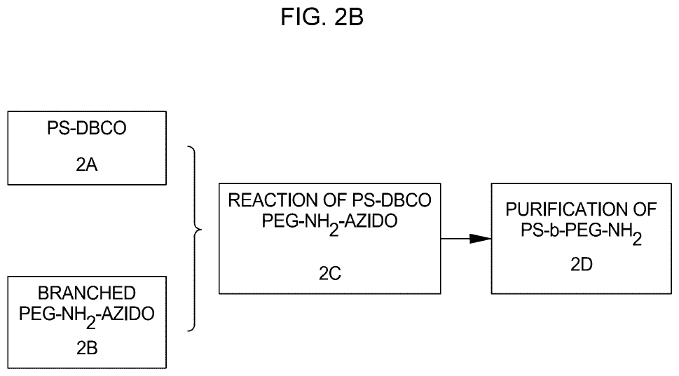

Now, with primary reference to FIG. 2A, a block flow diagram provides an overview of an illustrative polymer synthesis process of FIG. 1, Block 1A. Referring to FIG. 2A, Block 2A of the process, aminopolystyrene (“PS—NH₂) having linear formula (C₈H₈)ₙCH₅N, wherein the PS molecular weight can occur in a range of about 1.5 kDa to about 40 kDa, can be obtained from Polymer Source, Inc., PN P3965-SNH₂, comprising Formula I. Particular embodiments can include PS—NH₂ having a number averaged molecular weight of about 9.5 kDa.

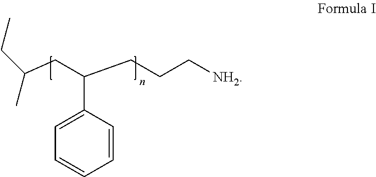

Now, referring to FIG. 2A, Block 2B of the polymer synthesis process, dibenzocylooctyne-N-hydroxysuccinimidyl ester (“DBCO-NHS Ester”) having liner formula C₂₃H₁₈N₂O₅ (CAS No.: 1353016-71-3) and having a molecular weight of 402.40 g/mol can be obtained from Click Chemistry Tools, PN A133-100 comprising Formula II.

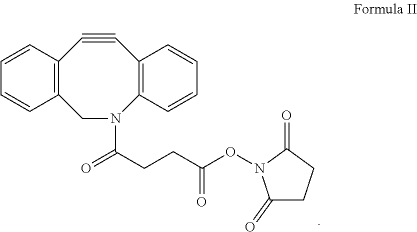 [SMILES: O=C(CCC(=O)N1Cc2ccccc2C#Cc2ccccc21)ON1C(=O)CCC1=O]

Now, referring to FIG. 2A, Block 2C of the polymer synthesis process, PS—NH₂ can be reacted with DBCO-NHS Ester to produce polystyrene-dibenzocylooctyne (“PS-DBCO”) comprising Formula III.

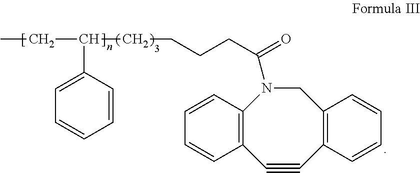

An example of a scalable procedure for the production of PS-DBCO can include thawing PS—NH₂ 20 mg for 15 minutes at room temperature (“RT”) and thawing DBCO—NHS-Ester 10 mg for 15 minutes (“min.”) at RT. Aliquot 1 mL of toluene (C₆H₅CH₃) (CAS No.: 108-88-3) to the PS—NH₂ 20 mg and vortex for 15 minutes at 500 RPM. Centrifuge DBCO at 1500 relative centrifugal force (“RCF”) (RCF═(RPM)²×1.118×10⁻⁵×r) for 30 seconds (“sec.”) at about 25° C. (about 77° F.). Aliquot 1 mL of toluene to the DBCO. Sonicate the DBCO for 5 min. in RT water. Transfer 1 mL of DBCO in toluene to PS—NH₂ in toluene. Vortex DBCO in PS—NH₂ mixture for 16 to 24 hours (“hr.”) at 500 RPM at RT.

Now, referring to FIG. 2A, Block 2D of the polymer synthesis process, an 8-arm polyethylene glycol-amine (“PEG₈₋ₐᵣₘ-NH₂”) comprising a multi-arm PEG derivative with amine groups at each terminal of the eight arms connected to one hexaglycerol core having linear formula R(O(CH₂CH₂O)nCH₂CH₂NH₂)₈ and a number averaged molecular weight of about 19.5 kDa can be obtained from Nanosoft Polymers, PN 2443, comprising Formula IV and further shown in Formula V.

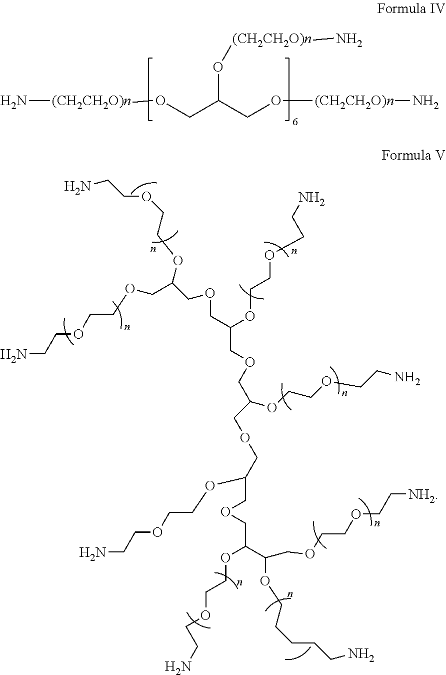

In particular embodiments, a 4-arm polyethylene glycol-amine, a 6-arm polyethylene glycol-amine, or an 8-armpolyethyleneglycol-amine, or combinations thereof, can also be utilized having corresponding PEG molecular weights of 10 kDa, 20 kDa, or 40 kDa.

Now, referring to FIG. 2A, Block 2E of the polymer synthesis process, an azido-d-polyethylene glycol 4-N-hydroxysuccinimidyl ester (“Azido-PEG₄-NHS Ester”) having a molecular weight of 388.37 g/mol (CAS No.: 944251-24-5) can be obtained from Click Chemistry tools PN AZ103-100 comprising Formula VI.

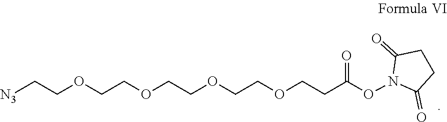 [SMILES: [N-]=[N+]=NCCOCCOCCOCCOCCC(=O)ON1C(=O)CCC1=O]

Now, referring to FIG. 2A, Block 2F of the polymer synthesis process, PEG₈ₐᵣₘ-NH₂ can be reacted with Azido-PEG4-NHS Ester to produce branched polyethylene glycol (“PEG-Amine-Azide”) comprising Formula VII.

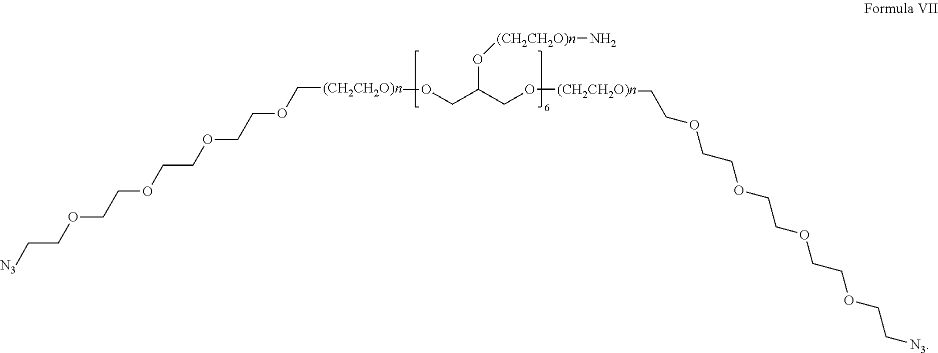

An example of a scalable protocol for the production of PEG-Amine-Azide can include thawing 40 mg of PEG₈₋ₐᵣₘ-NH₂ for 15 min. at RT and thawing 80 μl of Azido-PEG₄-NHS Ester (50 mM) for 15 min. at RT. Aliquot 400 μl of methanol (CAS NO.: 6756-1) to PEG₈₋ₐᵣₘ-NH₂ and vortex the PEG₈₋ₐᵣₘ-NH₂ for 15 min. at 500 RPM. Centrifuge Azido-PEG₄-NHS at 1500 RCF for 30 sec. at 25° C. (about 77° F.). Transfer the Azido-PEG4-NHS 80 μl to the 400 μl of PEG₈₋ₐᵣₘ-NH₂. Vortex for 16 to 24 hr. at 500 RPM at RT.

Now, referring to FIG. 2A, Block 2G of the polymer synthesis process, PS-DBCO obtained in Block 2C of the synthesis process can be purified by the following illustrative scalable procedure including transferring 400 μl of PS-DBCO to each of five centrifuge tubes. Aliquot 800 μl of methanol to each of the five centrifuge tubes. Mix PS-DBCO in methanol by inversion of each of the five centrifuge tubes. Centrifuge the PS-DBCO in methanol for 5 min. at 20,000 RCF at 15° C. to 25° C. (about 59° F. to about 77° F.). Decant the supernatant from each of five centrifuge tubes. Add 400 μl of toluene to each of the five centrifuge tubes. Place the five centrifuge tubes in a water bath at 37° C. (about 98.6° F.) for 2 min. Dissolve the PS-DBCO pellet in 400 μl toluene by mixing with a pipette. Add 800 μl of methanol to each of the five centrifuge tubes. Invert each centrifuge tube to mix contents. Centrifuge at 20,000 RCF for 5 min. at 15° to 25° C. (about 59° F. to about 77° F.). Decant the supernatant from each of five centrifuge tubes. Add 400 μl of toluene to each of the five centrifuge tubes. Place the five centrifuge tubes in a water bath at 37° C. (about 98.6° F.) for 2 min. Dissolve the PS-DBCO pellet in toluene by mixing with a pipette.

Now, referring to FIG. 2A, Block 2H of the polymer synthesis process, PS-DBCO obtained in Block 2G of the synthesis process can be reacted with PEG-Amine-Azide obtained in Block 2F of the polymer synthesis process to produce polystyrene-b-poly(ethylene glycol) amine (“PS-b-PEG-NH₂”) comprising Formula VIII.

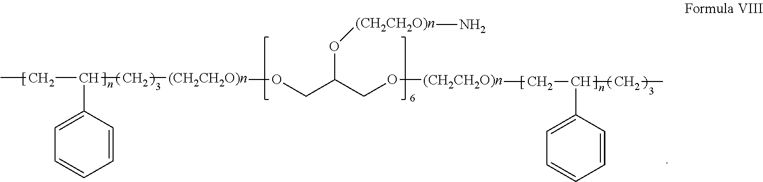

In the instant illustrative example of Block 2H, the PS-DBCO in toluene of all five centrifuge tubes from FIG. 2A, Block 2G and PEG-Amine-Azide in methanol obtained in Block 2F can be transferred to a 4 mL glass vial. Vortex the mixture of PS-DBCO and PEG-Amine-Azide at 500 RPM at RT for 16 to 24 hr. to produce PS-b-PEG-NH₂ comprising Formula VIII. The PS-b-PEG-NH₂ can comprise PS having molecular weights ranging from about 1.5 kDa to about 40 kDa and can comprise PEG having molecular weights ranging from about 10 kDa to about 40 kDa.

Referring to FIG. 2A, Block 2I of the synthesis process, the PS-b-PEG-NH₂ obtained in Block 2H can be purified and dried by the following illustrative scalable procedure including cooling hexane (CAS No.: 110-54-3) at −20° C. (about −4° F.) for 15 min. Aliquot approximately 1.25 mL of PS-b-PEG-NH₂ obtained in Block 2I of the synthesis process into a 15 mL centrifuge tube. Slowly add 12 mL of hexane to the centrifuge tube containing PS-b-PEG-NH₂. Mix the PS-b-PEG-NH₂ in toluene/hexane by gently tilting the centrifuge tube 10 to 15 times. Place the centrifuge tube at −20° C. (about −4° F.) for 5 min. to cool the PS-b-PEG-NH₂ in toluene/hexane. Decant the supernatant from the PS-b-PEG-NH₂ pellet. Aliquot 1 mL of tetrahydrofuran to the centrifuge tube. Place the centrifuge tube in a water bath at the 37° C. (about 98.6° F.) for 2 min. Dissolve the PS-b-PEG-NH₂ pellet by mixing with a pipette. Slowly add 6 mL of cold hexane to the centrifuge tube. Gently tilt the centrifuge tube 10 to 15 times until PS-b-PEG-NH₂ precipitates as flakes are visualized with clear supernatant. Place the centrifuge tube at −20° C. (about −4° F.) for 5 min. Decant the supernatant from the PS-b-PEG-NH₂ pellet. Place the centrifuge tube containing the PS-b-PEG-NH₂ pellet at RT for 24 hr. to remove excess solvents and to obtain a dry PS-b-PEG-NH₂. Store at −20° C. (about −4° F.).

Now, with primary reference to FIG. 2B, a block flow diagram provides an overview of an illustrative polymer synthesis process of FIG. 1, Block 1A. Referring to FIG. 2B, Block 2A of the process, polystyrene-DBCO (“PS-DBCO) can have a PS molecular weight occurring in a range of about 1.5 kDa to about 40 kDa, can be obtained from Akina Inc., 3495 Kent Avenue, West Lafayette, IN 47906 USA. Particular embodiments can include PS-DBCO having a number averaged molecular weight of about 9.5 kDa comprising Formula IX.

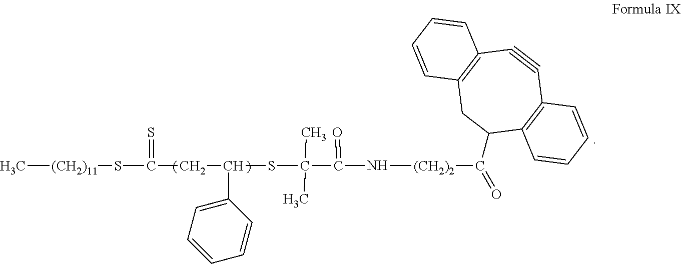

Now, referring to FIG. 2, Block 2B of the polymer synthesis process, an 8-arm polyethylene glycol-7amine-1 azide (“PEG₈₋ₐᵣₘ-NH₂-Azide”) comprising a multi-arm PEG derivative with 7 amine groups at each terminal of the eight arms and 1 azide group connected to one tripentaerythritol core having linear formula R(O(CH₂CH₂O)nCH₂N₃)((CH₂CH₂O)NH₂)₇ and a number averaged molecular weight of about 19.5 kDa can be obtained from Creative Peg Works, P.O. Box 16863, Chapel Hill, NC 27516, USA, Product No. CPW-8812TP, comprising Formula X.

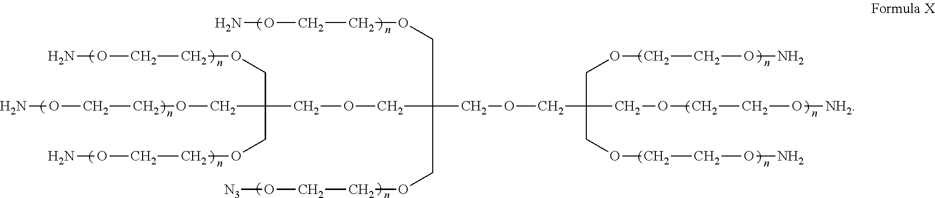

Now, referring to FIG. 2B, Block 2C of the polymer synthesis process, PS-DBCO obtained in FIG. 2B, Block 2A of the synthesis process can be reacted with PEG₈₋ₐᵣₘ-NH₂-Azide obtained in FIG. 2B, Block 2B of the polymer synthesis process to produce polystyrene-b-poly (ethylene glycol) amine (“PS-b-PEG-NH₂”) comprising Formula XI.

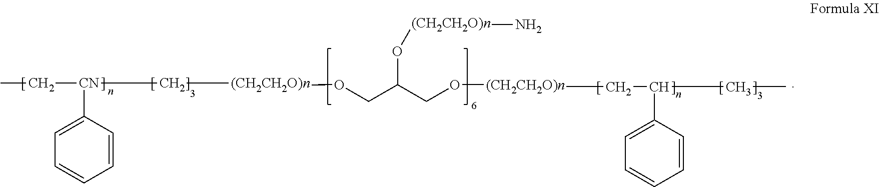

In the instant illustrative example, 20 mg of PS-DBCO in 2 mL Toluene obtained in FIG. 2B, Block 2A can be combined with 80 mg of PEG-Amine-Azide in 800 μL of methanol obtained in FIG. 2B, Block 2B and be transferred to a 4 mL glass vial. Vortex the mixture of PS-DBCO and PEG-Amine-Azide at 500 RPM at RT for 16 to 24 hr to produce PS-b-PEG-NH₂ comprising Formula XI. The PS-b-PEG-NH₂ can comprise PS having molecular weights ranging from about 1.5 kDa to about 40 kDa and can comprise PEG having molecular weights ranging from about 10 kDa to about 40 kDa.

Referring to FIG. 2B, Block 2D of the synthesis process, the PS-b-PEG-NH₂ obtained in FIG. 2B, Block 2C can be purified and dried by the following illustrative scalable procedure including cooling hexane (CAS No.: 110-54-3) at −20° C. (about −4° F.) for 15 min. Aliquot approximately 1.25 mL of PS-b-PEG-NH₂ obtained in Block 2I of the synthesis process into a 15 mL centrifuge tube. Slowly add 12 mL of hexane to the centrifuge tube containing PS-b-PEG-NH₂. Mix the PS-b-PEG-NH₂ in toluene/hexane by gently tilting the centrifuge tube 10 to 15 times. Place the centrifuge tube at −20° C. (about −4° F.) for 5 min. to cool the PS-b-PEG-NH₂ in toluene/hexane. Decant the supernatant from the PS-b-PEG-NH₂ pellet. Aliquot 1 mL of tetrahydrofuran to the centrifuge tube. Place the centrifuge tube in a water bath at the 37° C. (about 98.6° F.) for 2 min. Dissolve the PS-b-PEG-NH₂ pellet by mixing with a pipette. Slowly add 6 mL of cold hexane to the centrifuge tube. Gently tilt the centrifuge tube 10 to 15 times until PS-b-PEG-NH₂ precipitates as flakes are visualized with clear supernatant. Place the centrifuge tube at −20° C. (about −4° F.) for 5 min. Decant the supernatant from the PS-b-PEG-NH₂ pellet. Place the centrifuge tube containing the PS-b-PEG-NH₂ pellet at RT for 24 hr. to remove excess solvents and to obtain a dry PS-b-PEG-NH₂. Store at −20° C. (about −4° F.).

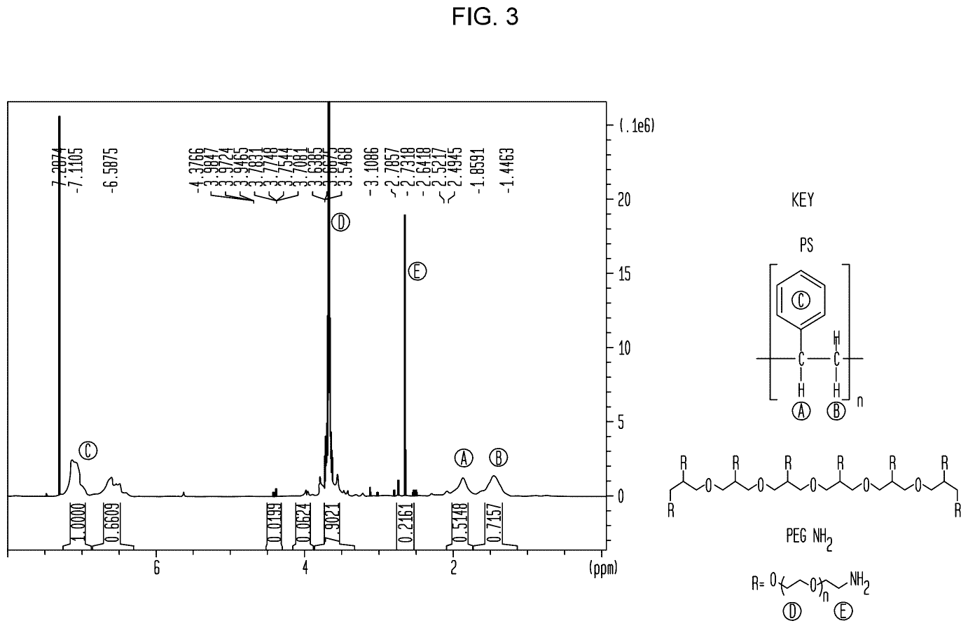

Now, referring primarily to FIG. 3, the structure of the PS-b-PEG-NH₂ obtained in FIG. 2A, Block 2I and obtained in FIG. 2B, Block 2D of the synthesis process can be analyzed using nuclear magnetic resonance (“NMR”). The NMR spectrum shown in FIG. 3 validates the molecular structure obtained by the polymer synthesis process shown in FIG. 2A, Blocks 2A through 2I, and in FIG. 2B, Blocks 2A through 2D, and above disclosed polymer synthesis procedures, is purified PS-b-PEG-NH₂ comprising respectively Formula VIII and Formula XI.

The illustrative example of the polymer PS-b-PEG-NH₂ obtained in FIG. 2A, Block 2I and FIG. 2B, Block D is not intended to preclude embodiments of the PS-b-PEG including functional groups other than NH₂. Other functional groups can include as examples, one or more of: acrylate, maleimide, vinylsulfone, azide, biotin, carboxyl, thiol, alkyne, hydrazide, N-hydroxysuccinimide ester, and nitrophenyl carbonate, and combinations thereof, or embodiments including other similar or equivalent polymers including one or more functional groups.

### Formation of Polymer Encapsulated Nanoparticles.

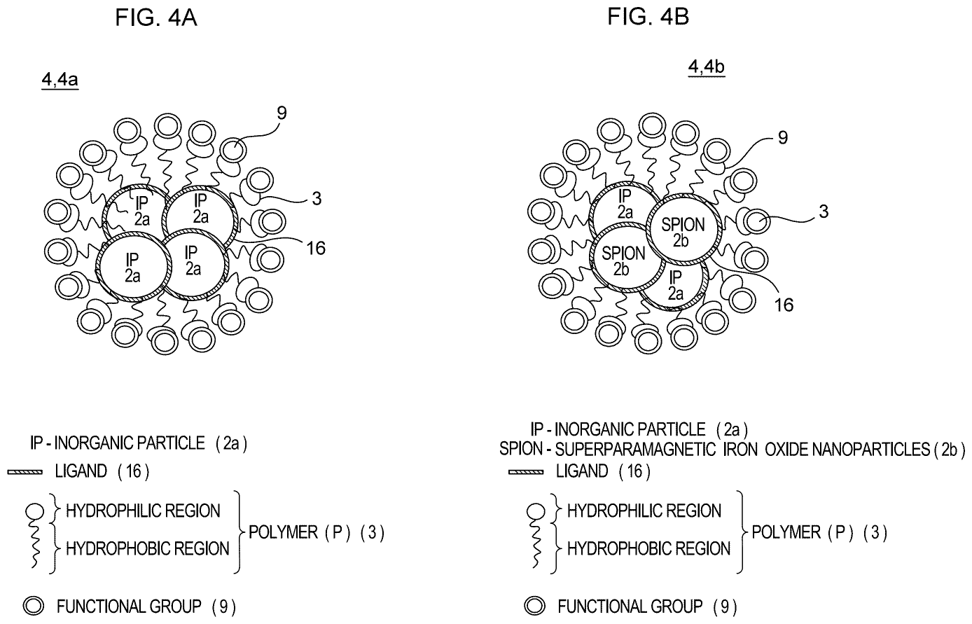

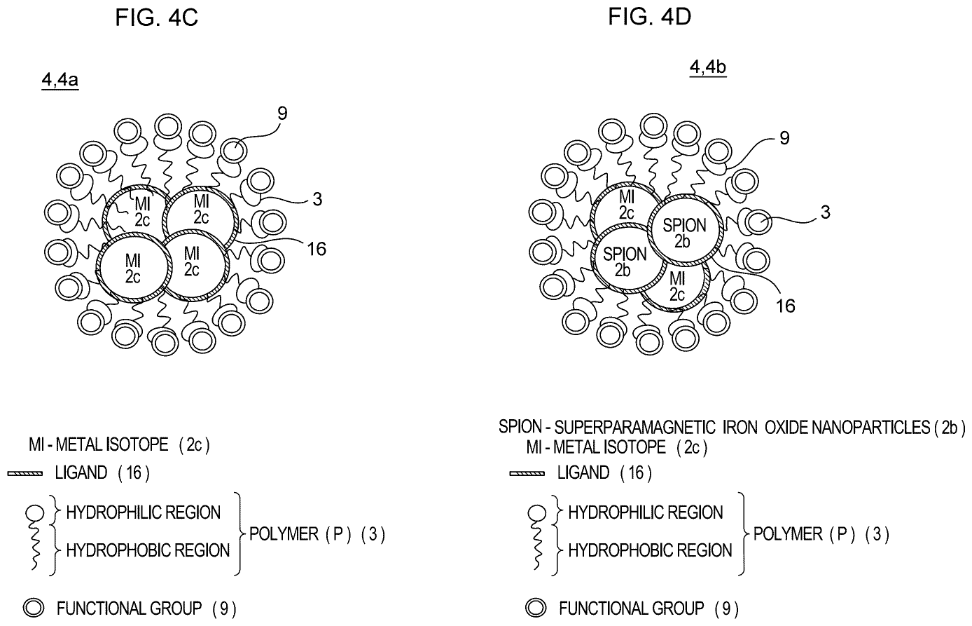

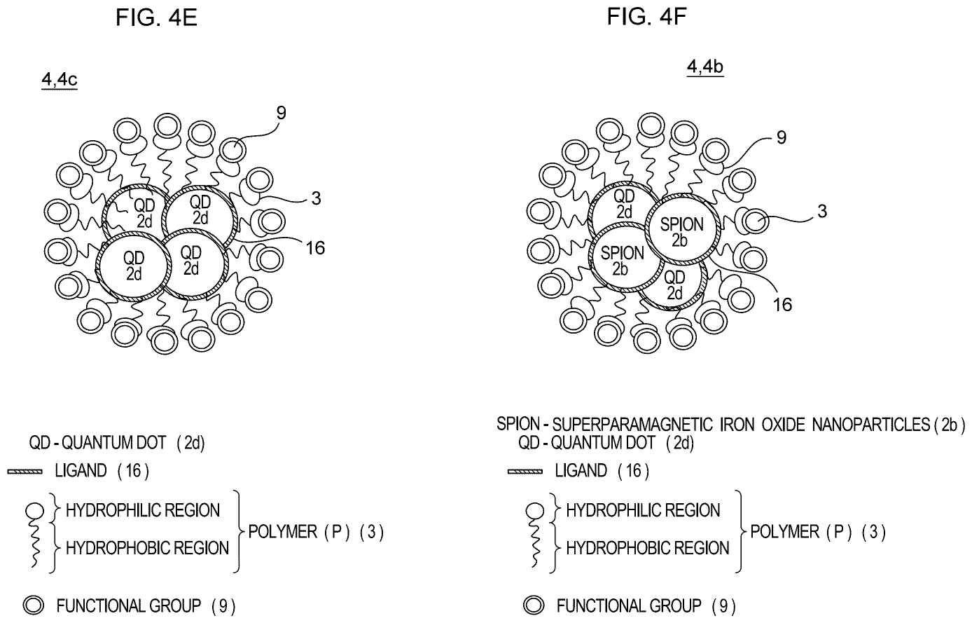

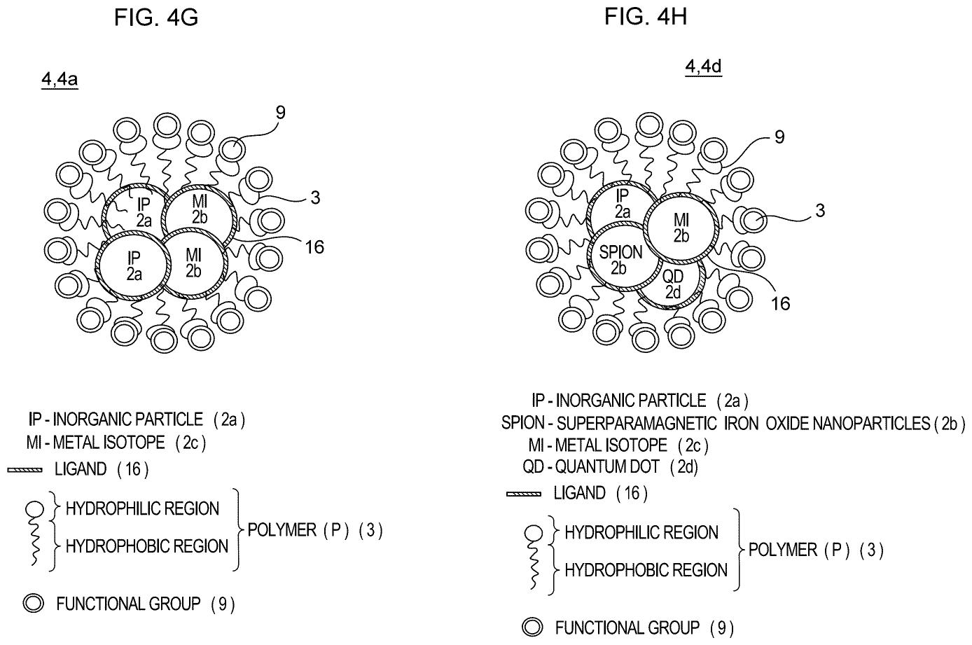

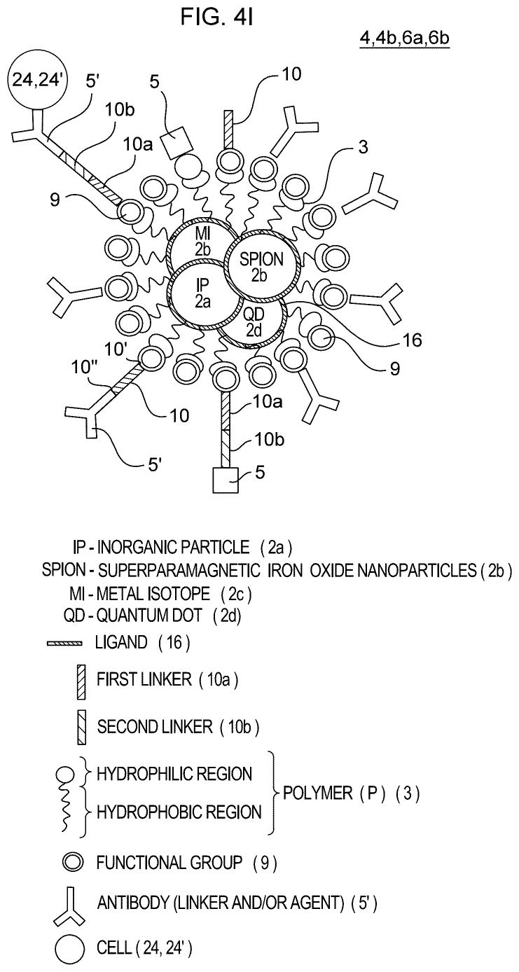

Now, with primary reference to FIG. 4A which illustrates a particular embodiment of PENP (4) including one or more inorganic particles (2a) (“IP”) encapsulated in a polymer (“P”) (3) having a hydrophobic region which can associate or coordinate with the one or more in organic particles (2a) and a hydrophilic region including a functional group (9) which can associate with an aqueous environment, and in particular embodiments, the polymer (P) (3) can comprise PS-b-PEG-NH₂ obtained in FIG. 2A, Block 2I or FIG. 2B, Block D, which by solution based association can produce polymer encapsulated nanoparticles including one or more inorganic particles (2a) (also referred as an “IsoDot”) (4a). The term “inorganic particle (IP) (2a)” as used in embodiments of the invention means one or more of: metal-based particles including metal oxides, metal halides, doped metal, metal isotopes (“MI”); non-metal isotopes; quantum dots (“QD”); Perovskite quantum dots; and supramagnetic iron oxide nanoparticles (“SPION”). The term “particle” broadly encompasses an atom or nanoparticles each including hundreds or thousands atoms.

Now, with primary reference to FIG. 4B which illustrates a particular embodiment of PENP (4) including and one or more SPION (2b) encapsulated in a polymer (“P”) (3) having a hydrophobic region which can associate or coordinate with the one or more SPION (2b) (also referred to as a “MagDot”), and optionally includes one or more inorganic particles (2a), and a hydrophilic region including a functional group (9) which can associate with an aqueous environment, and in particular embodiments, the polymer (P) (3) can comprise PS-b-PEG-NH₂ obtained in FIG. 2A, Block 2I or FIG. 2B, Block 2D, which by solution based association can produce PENP (4) including only one or more SPION (2b) or one or more SPION and one or more IP (2a). The term “SPION (2b)” as used in embodiments of the invention means a superparamagnetic iron oxide nanoparticles of maghemite having an empirical formula of Fe₂O₃, γ—Fe₂O₃ and a molar mass 159.69 g/mol (CAS No. 1309-37-1) or magnetite having an empirical formula of Fe₃O₄ and a molar mass of 231.53 g/mol (CAS NO. 1317-61-9), or combinations thereof, with a core ranging from about 5 nm to about 30 nm in diameter.

Now, with primary reference to FIG. 4C which illustrates a particular embodiment of PENP (4) including and one or more metal isotopes (2b) encapsulated in a polymer (“P”) (3) having a hydrophobic region which can associate or coordinate with the one or more metal isotopes (2c) and a hydrophilic region including a functional group (9) which can associate with an aqueous environment, and in particular embodiments, the polymer (P) (3) can comprise PS-b-PEG-NH₂ obtained in FIG. 2A, Block 2I or FIG. 2B, Block 2D, which by solution based association can produce PENP (4) including one or more metal isotopes (2b). The term “metal isotope (2c) (MI)” as used in embodiments of the invention means each of two or more forms of the same element that contain equal numbers of protons but different numbers of neutrons in their nuclei, and hence differ in relative atomic mass but not in chemical properties.

Now, with primary reference to FIG. 4D which illustrates a particular embodiment of PENP (4) including one more MI and one more SPION encapsulated in a polymer (“P”) (3) having a hydrophobic region which can associate or coordinate with the one or more metal isotopes (2c) and the one or more SPION and a hydrophilic region including a functional group (9) which can associate with an aqueous environment, and in particular embodiments, the polymer (P) (3) can comprise PS-b-PEG-NH₂ obtained in FIG. 2A, Block 2I or FIG. 2B, Block 2D, which by solution based association can produce PENP (4) including one or more metal isotopes (2b) and the one or more SPION.

Now, with primary reference to FIG. 4E which illustrates a particular embodiment of PENP (4) including one more quantum dots (2d) encapsulated in a polymer (“P”) (3) having a hydrophobic region which can associate or coordinate with the one or more quantum dots (2d) and a hydrophilic region including a functional group (9) which can associate with an aqueous environment, and in particular embodiments, the polymer (P) (3) can comprise PS-b-PEG-NH₂ obtained in FIG. 2A, Block 2I or FIG. 2B, Block 2D, which by solution based association can produce PENP (4) including one or more quantum dots (2d). The term “quantum dot (2d)” as used in embodiments of this invention means semiconductor nanoparticles with optical and electronic properties that can change as a function of both size and shape and without limitation to the forgoing broad definition includes as an example perovskite quantum dots.

Now, with primary reference to FIG. 4F which illustrates a particular embodiment of PENP (4) including one more quantum dots (2d) and one or more SPION (2b) encapsulated in a polymer (“P”) (3) having a hydrophobic region which can associate or coordinate with the one or more quantum dots (2d) and the one or more SPION (2b) and a hydrophilic region including a functional group (9) which can associate with an aqueous environment, and in particular embodiments, the polymer (P) (3) can comprise PS-b-PEG-NH₂ obtained in FIG. 2A, Block 2I or FIG. 2B, Block 2D, which by solution based association can produce PENP (4) including one or more quantum dots (2d) and one or more SPION.

Now, with primary reference to FIG. 4G which illustrates a particular embodiment of PENP (4) including one or more inorganic particles (2a), one or more metal isotopes (MI) and one more quantum dots (2d) encapsulated in a polymer (“P”) (3) having a hydrophobic region which can associate or coordinate with the one or more inorganic particles (2a), one or more metal isotopes (MI) and one more quantum dots (2d) and a hydrophilic region including a functional group (9) which can associate with an aqueous environment, and in particular embodiments, the polymer (P) (3) can comprise PS-b-PEG-NH₂ obtained in FIG. 2A, Block 2I or FIG. 2B, Block 2D, which by solution based association can produce PENP (4) including one or more inorganic particles (2a), one or more metal isotopes (MI) and one more quantum dots (2d).

Now, with primary reference to FIG. 4H which illustrates a particular embodiment of PENP (4) including one or more inorganic particles (IP) (2a), one or more metal isotopes (MI) (2b), one more quantum dots (QD) (2d), and one or more SPION (2b) encapsulated in a polymer (“P”) (3) having a hydrophobic region which can associate or coordinate with the one or more inorganic particles (2a), one or more metal isotopes (MI) and one more quantum dots (2d) and one or more SPION (2b) and a hydrophilic region including a functional group (9) which can associate with an aqueous environment, and in particular embodiments, the polymer (P) (3) can comprise PS-b-PEG-NH₂ obtained in FIG. 2A, Block 2I or FIG. 2B, Block 2D, which by solution based association can produce PENP (4) including one or more inorganic particles (2a), one or more metal isotopes (MI) and one more quantum dots (2d) and one or more SPION (2b).

Now, with primary reference to FIG. 4I, a numerous and wide variety of agents (5) can be conjugated to particular embodiments of PENP (4) which can include one or more inorganic particle (2a) (IP), one or more SPION (2b), one or more metal ion (2c) and one or more quantum dot (2d), and combinations thereof, to produce a numerous and wide variety of IsoDots (4a) and/or MagDots (4b) using the functional group (9) of the polymer (3), and in particular embodiments, the amine afforded by PS-b-PEG-NH₂. The example of FIG. 4I illustrates that one or more agents (5) can be conjugated to embodiments of PENP (4) including embodiments of IsoDots (4a) and/or MagDots (4b) by activating the functional group (9), including as illustrative examples: one or more linkers (10, 10a, 10b), a polyethylene glycol, a fluorescent probe, an aptamer, a vitamin, a radioactive isotope, a contrast media, a surface charge modifier, a lectin, a protein, a peptide, a cell surface receptor, a cell coat, and combinations thereof. Specifically, in particular embodiments the functional group (9) can be utilized to directly or indirectly through one or more linkers (10, 10a, 10b) couple an antibody or antibody fragment (Ab) (5′) to produce PENP antibody conjugates (6) including as illustrative examples IsoDots-Ab (6a) and MagDots-Ab (6b).

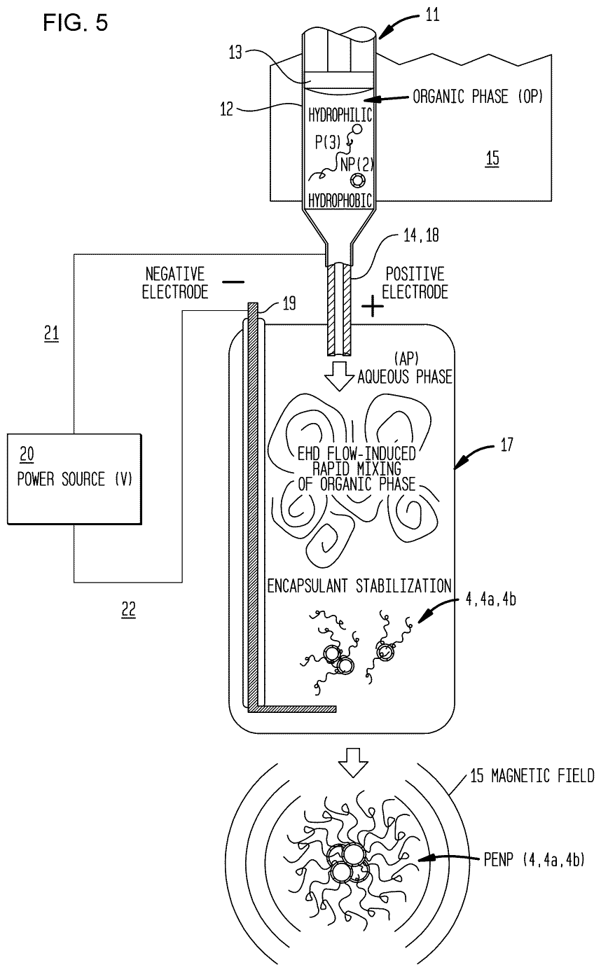

Now, with primary reference to FIG. 5, scalable, solution-based production of PENP (4) encompassed by the invention, including, but not necessarily limited to, MagDots (4b) or IsoDots (4a), can be prepared by conventional self-assembly, flash nanoprecipitation (FNP) comprising rapid turbulent mixing generated by high velocity flows, as described by Yanjie Zhang, Aaron R. Clapp, RSC Advances, Issue 89, 2014 “Preparation of quantum dot-embedded polymeric nanoparticles using flash nanoprecipitation”, or by rapid mixing induced by electrohydrodynamics (“EHD”): EHD mixing mediated-nanoprecipitation (“EHD-NP”). Kil Ho Lee, Guolingzi Yang, Barbara E. Wyslouzil and Jessica O. Winter, ACS Appl. Polym. Mater. 2019, 1, 4, 691-700, each incorporated by reference herein.

The illustrative EHD mixing system of FIG. 5 can include a syringe (11) having a syringe barrel (12) fitted with a sliding syringe plunger (13), and a syringe needle (14). A syringe pump (15) configured drive the syringe plunger (13) to deliver an organic phase of water-miscible nonpolar aprotic solvents (“OP”) including solubilized (P) (3) and NP (4) whether MI (2a) and/or SPIONS (2b) (collectively the “inorganics”) at a predetermine volumetric flow rate into an aqueous phase (“AP”). The illustrative example of a syringe (11) and a syringe driver (15) is not intended to preclude other appliances useful in delivering the OP into the AP at a predetermined volumetric flow.

The concentration of inorganics per unit volume of the OP can be about 0.1 volume to volume (“v/v”) to about 0.5 v/v. The concentration of P (3) to MI (2a) and/or SPION (2b) in the OP by mass can be about 1:1 to about 4:1. The amount of P (3) can be adjusted to obtain a PENP (4) having a substantially uniform hydrodynamic diameter (“HD”) that occurs in size range of about 20 nanometers (“nm”) to about 500 nm. In embodiments in which the MI (2a) and/or the SPIONS (2b) are passivated with a ligand (16), the ligand mass relative to the total inorganics mass can be about 20% to 40% by mass. A ligand mass percent greater than 40% can interfere with the assembly of the PENP (4).

A non-electrically conductive container (17) can hold the AP, typically distilled or deionized water. A positive electrode (18) and a negative electrode (19) can be introduced about 1 cm apart in the AP held by the non-electrically conductive container (17). In particular embodiments the syringe needle (14) can, if electrically conductive, act as the positive electrode (18). A voltage source (20) can supply a voltage (“V”) to the positive terminal (18) to generate an electrical field between the positive electrode (18) and negative electrode (19) in the AP. The syringe plunger (13) can be driven to introduce the OP into the AP at a consistent flow rate of about 8 mL h⁻¹ to about 15 mL h⁻¹. Voltage (V) can be adjusted between about −1 kilovolt (“kV”) and about −2.5 kV. The electric field can generate a fine dispersion of OP in the AP to produce PENP (4), MagDots (4b), or IsoDots (4a) of substantially uniform size. The resulting size of the PENP (4), IsoDots (4a), MagDots (4b) can increase with increasing V and/or concentration of inorganics per unit volume of the OP. The resulting MagDots (4b) can be subsequently isolated by influence of a magnetic field (15).

### IsoDots-IP and IsoDots-MI.

Now, with primary reference to FIGS. 4A through 4I and 5, IsoDots (4a) can encapsulate a wide range of metal isotope nanoparticles (MI) (2a) and non-metal isotope nanoparticles including, as illustrative examples (listed lowest to highest atomic mass):
As\hat{ }33 Arsenic: As-75
Se\hat{ }34 Selinium: Se-74, Se-76, Se-77, Se-78, Se-80, Se-82
Br\hat{ }35 Bromine: Br-79, Br-81
Y\hat{ }39 Ytrium: Y-89
Zr\hat{ }40 Zirconium: Zr-90, Zr-91, Zr-92, Zr-94, Zr-96
Mo\hat{ }42 Molybdenum: Mo-92, Mo-94, Mo-95, Mo-96, Mo-97, Mo-98, Mo-100
Ru\hat{ }44 Ruthenium: RE-96, RE-98, RE-99, RE-100, RE-101, RE-102, RE-104
Rh\hat{ }45 Rhodium: Rh-103
Pd\hat{ }46 Palladium: Pd-102, Pd-104, Pd-105, Pd-106, Pd-108, Pd-110
Ag\hat{ }47 Silver: Ag-107, Ag-109
Cd\hat{ }48 Cadmium: Cd-106, Cd-108, Cd-101, Cd-111, Cd-112, Cd-113, Cd-114, Cd-116
In\hat{ }49 Indium: In-113, In-115
Sn\hat{ }50 Tin: Sn-112, Sn-114, Sn-115, Sn-116, Sn-117, Sn-118, Sn-119, Sn-120, Sn-122, Sn-124
Sb\hat{ }51 Antimony: Sb-121, Sb-123
Te\hat{ }52 Tellurium: Te-120, Te-122, Te-123, Te-124, Te-125, Te-126, Te-128, Te-130
I\hat{ }53 Iodine: I-127
Cs\hat{ }55 Caesium: Cs-133
Ba\hat{ }56 Barium: Ba-130, Ba-132, Ba-134, Ba-135, Ba-136, Ba-137, Ba-138
La\hat{ }57 Lanthanum: La-138, La-139
Ce\hat{ }58 Cerium: Ce-136, Ce-133, Ce-140, Ce-142
Pr\hat{ }59 Praseodymium: Pr-141
Nd\hat{ }60 Neodymium: Nd-142, Nd-143, Nd-144, Nd-145, Nd-146, Nd-148, Nd-150
Sm\hat{ }62 Samarium: Sm-144, Sm-147, Sm-148, Sm-149, Sm-150, Sm-152, Sm-154
Eu\hat{ }63 Europium: Eu-151, Eu153
Gd\hat{ }64 Gadolinium: Gd-152, Gd-154, Gd-155, Gd-156, Gd-157, Gd-158, Gd-160
Tb\hat{ }65 Terbium: Tb-159
Dy\hat{ }66 Dyprosium: Dy-156, Dy-158, Dy-160, Dy-161, Dy-162, Dy-163, Dy-164
Ho\hat{ }67 Holmium: Ho-165
Er\hat{ }68 Erbium: Er-162, Er-164, Er-166, Er-167, Er-168, Er-170
Tm\hat{ }69 Thulium: Tm-169
Yb\hat{ }70 Ytterbium: Yb-168, Yb-170, Yb-171, Yb-172, Yb-173, Yb-174, Yb-176
Lu\hat{ }71 Lutetium: Lu-175, Lu-176
Hf\hat{ }72 Hafnium: Hf-174, Hf-176, Hf-177, Hf-178, Hf-179, Hf-180
Ta\hat{ }73 Tantalum: Ta-180, Ta-181
W\hat{ }74 Tungsten: W-180, W-182, W-183, W-184, W-186
Re\hat{ }75 Rhenium: Re-185, Re-187
Pt\hat{ }78 Platinum: Pt-190, Pt-192, Pt-194, Pt-195, Pt-196, Pt-198
Au\hat{ }79 Gold: Au-197
Hg\hat{ }80 Mercury: Hg-196, Hg-198, Hg-199, Hg-200, Hg-201, Hg-202, Hg-204
Pb\hat{ }82 Lead: Pb-204, Pb-206, Pb-207, Pb-208
Bi\hat{ }83 Bismuth: Bi-209

IsoDots can be formed to encapsulate metal and non-metal isotope variants that have the same number of protons but a different number of neutrons, or can be formed to encapsulate different metal or non-metal isotopes. Each IsoDot (4, 4a) can load one or a plurality of metal or non-metal isotope atoms or metal isotope particles each including hundreds or thousands of metal isotope atoms. The non-metal or metal particles can range in size from about 2 nanometers to about 100 nanometers with the resulting IsoDots (4a) having a hydrodynamic diameter of about 20 nanometers (“nm”) to about 500 nm.

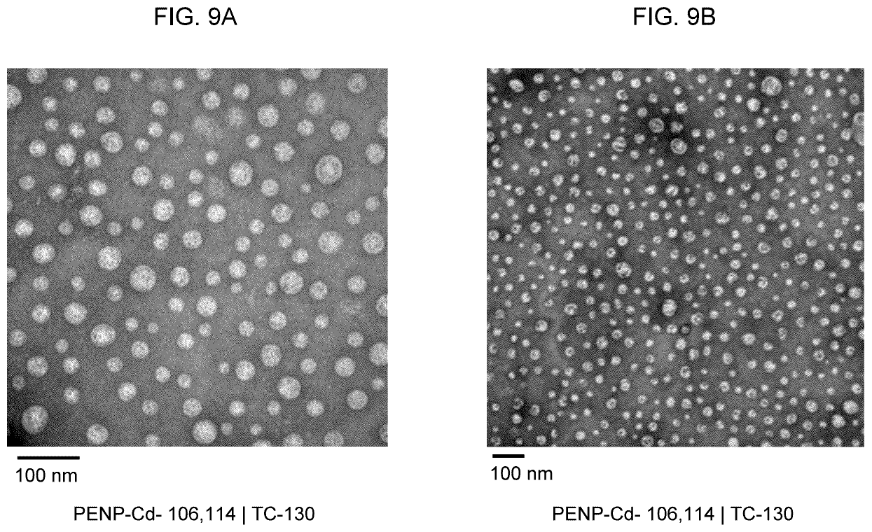

In particular embodiments, IsoDots (4, 4a) can be formed to encapsulate only metal or non-metal isotope nanoparticles that naturally occur with atoms of the same atomic mass. As an illustrative example, pure Au isotope nanoparticle(s) having only atoms of an atomic mass of 197. FIG. 9 is a micrograph of IsoDots-AU 197. Similarly, IsoDots (4, 4a) can be formed with other pure metal isotope nanoparticles each having only atoms of the same atomic mass, such as isotopes of Rh, Y, Cs, Pr, Tb, and Ho.

In other particular embodiments, IsoDots (4, 4a) can include metal isotope metal particles that are not isotopically pure by nature but can be isotopically enriched to a suitable level of purity for embodiments of the invention. As illustrative example, metal isotopes can be obtained through Trace Sciences International 501 Silverside Road, Suite 342, Wilmington, Delaware 19809 USA. As an illustrative example: Te can be purified to separate Te 120, Te 122, Te 123, Te 124, Te 125, Te 126, Te 128, and Te 130 to afford certain Te isotopes sufficiently enriched for embodiments of the invention.

In other particular embodiments, IsoDots (4, 4a) can include metal or non-metal isotope nanoparticles of one element including two or more isotopes. As illustrative examples, Ag nanoparticles can include Ag 107 and Ag 109, and Pt nanoparticles can include Pt 190, Pt 192, Pt 194, Pt 195, and Pt 198.

In other embodiments, an IsoDot (4, 4a) can include metal or non-metal isotope nanoparticles where one element(s) is isotopically pure and the other element(s) include two or more isotopes. As an illustrative example: As has one isotope As 75 and In can include two isotopes In 113 and In 115.

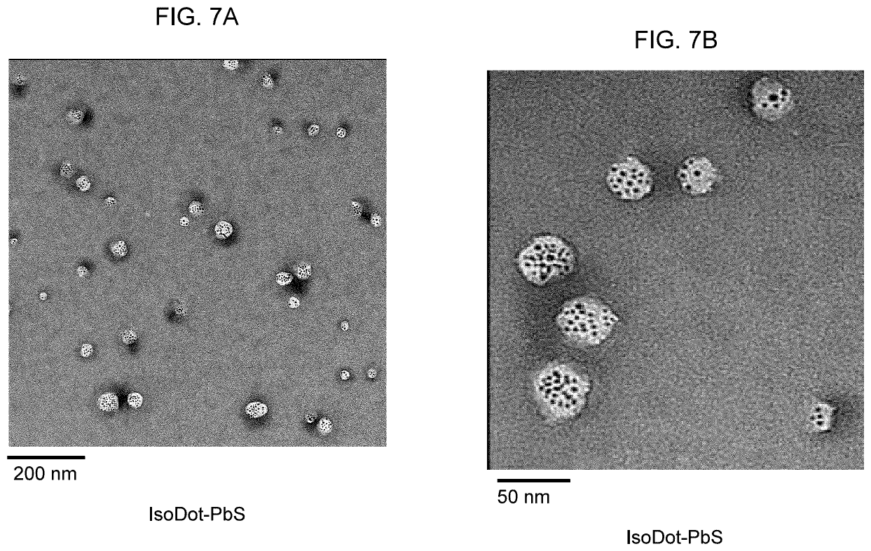

In other embodiments, an IsoDot (4, 4a) can include metal or non-metal isotope nanoparticles including two or more elements where each of the elements includes a plurality of isotopes. As an illustrative example an IsoDot (4, 4a) can include: Cd including one or more of Cd 101, Cd 106, Cd108, Cd 111, Cd112, Cd113, Cd114, Cd 116; Te including one or more of Te 120, Te 122, Te 123, Te 124, Te 125, Te 126, Te 128 and Te 123; and Se including one or more of Se 74, Se 76, Se 77, Se 78, Se 80 and Se 82. FIGS. 7A and 7B are micrographs of IsoDots-Cd114/Te130.

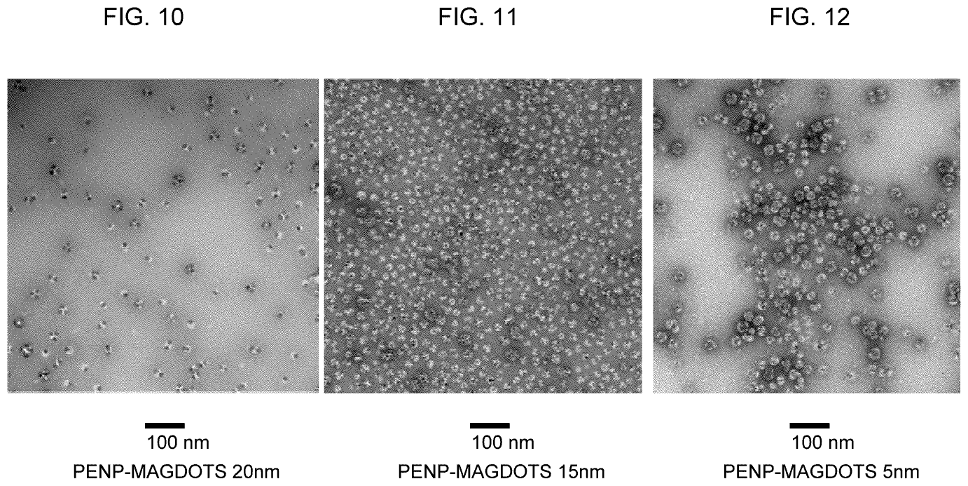

In other embodiments, a MagDot (4, 4b) can be formed to encapsulate one or more SPION wherein the SPION particles can be selected within the range of about 5 nm to about 100 nm and as illustrative examples can include SPION particles of 5 nm, 15 nm or 20 nm. FIGS. 10, 11 and 12 are micrographs of MagDots including SPION particles of 5 nm, 15 nm or 20 nm respectively. MagDots (4, 4b) can further include one or more inorganic particles (IP) including non-metal isotopes, MI or QD.

In other embodiments, a QDot (4, 4c) can be formed to encapsulate one or more QD. The encapsulated QDs can comprise only one wavelength emission in the range of about 400 nm to about 1600 nm or can comprise different QDs each having a wavelength emission within the range of about 400 nm to about 1600 nm as described in U.S. Pat. No. 12,370,266, hereby incorporated by reference herein.

In other embodiments, a MultiDot (4, 4d) can be formed to encapsulate in various combinations one or more of: IP, MI, QD, and/or SPION as above described

Embodiments of purified IP (2a), SPION (2b), MI (2c) or QD (2d) nanoparticles can be bare or can be capped to control nanoparticle size and/or to prevent agglomeration. As an illustrative example, QD (2d) synthesized by prototypical hot-injection method can be capped with a ligand (16), such as, oleylamine and oleic acid after QD purification. ¹H NMR spectroscopy analysis evidence that ligand binding can be highly dynamic, and that oleylamine selectively binds to the surface as oleylammonium bromide in an NC(X)₂ binding motif. Only in the presence of excess oleylamine added after purification does oleic acid bind to the surface, in the form of oleylammonium oleate. Protesescu L, Yakunin S, Bodnarchuk M I, Krieg F, Caputo R, Hendon C H, Yang R X, Walsh A, Kovalenko M V, Nano Lett. 2015, 15, 3692-3696, hereby incorporated by reference herein. While the use of oleylamine and oleic acid as a capping ligand (16) is suitable for embodiments of the invention, this is not intended to preclude embodiments using other capping ligand(s) (16), as examples, trioctylphosphine oxide, L-histidine, chitosan, polyvinyl alcohol, polyvinylpyrrolidone or combinations thereof.

Typically, bare or capped nanoparticles can reach a required level of water solubility and biocompatibility by surrounding the nanoparticle (2a, 2b, 2c, 2d) to produce PENP (4). A P (3) suitable for use with embodiments of the invention can include various embodiments of PS-b-PEG-NH₂ obtained in Block 2I of the synthesis process, above described. Embodiments of PENP (4) can be produced through the use of various combinations of one or more of: IP (2a), SPION (2b), MI (2c), QD (2d), capping ligand (16), and PS-b-PEG-NH₂ having PS molecular weights ranging from about 1.5 kDa to about 40 kDa and PEG having molecular weights ranging from about 10 kDa to about 40 kDa.

An illustrative example of a scalable method of production PENP (4), including but not necessarily limited to, IsoDots (4a), Mag Dots (4b), QDots (4c) or MultiDots (4d) by EHD can include one or more of: constitute PS-b-PEG-NH₂ obtained in FIG. 2, Block 2I at 10 mg/mL into a first 1.5 mL tube. Introduce 240 μl of the desired ID (2a) at 5 mg/mL into a second 1.5 mL tube. Transfer 480 μl of acetone/methanol (60/40) to the ID (2a) in the second 1.5 mL tube. Centrifuge the second 1.5 mL tube containing the ID (2a) at 7000 RCF for 1 min, and then remove supernatant with a 200 μL pipette. Transfer 480 μL of anhydrous tetrahydrofuran (“THF”) to the second 1.5 mL tube containing the IP, MI, QD nanoparticles and mix thoroughly with a pipette. Introduce into a fresh 1.5 mL centrifuge tube 240 μl of THE, 240 μl IP, MI, QD nanoparticles in THE, and 120 μl of the solubilized PS-b-PEG-NH₂ to produce the OP for EHD.

Particular embodiments, EHD can be performed by cleaning the EHD mixing system syringe (11) three times with THF. Load about 0.6 mL of the OP into the syringe barrel (12). Mix the inorganics in the OP thoroughly while loading the syringe (11). Attach the syringe (11) to the syringe pump (15) and set the syringe pump (15) to generate a flow rate of the OP containing the inorganics from the syringe needle (14) in the range of about 11.00 mL/hr. to about 14.00 mL/hr. In particular embodiment the flow rate can be about 12.5 mL/hr. In particular embodiment the flow rate can be about 12.5 mL/hr. Prime the syringe needle (14) until a drop of the OP forms at the end of the syringe needle (14). Twice rinse a 20 mL glass vial (17) with distilled or deionized water (individually or collectively “DI water”). Introduce about 10 mL of DI water into the 20 mL glass vial. Submerge the syringe needle (14) into the 20 mL glass vial (17). Clean the negative electrode (19) by submerging in THE, wipe, and rinse with DI water. Place the negative electrode (19) into the AP contained in the 20 mL glass vial. Place the positive electrode (18) into the AP contained in the 20 mL glass vial (17). In particular embodiments the syringe needle (14), if electrically conductive, can act as the positive electrode (18). Observe that the positive electrode (18), the negative electrode (19), and the syringe needle (14) do not contact. Connect the positive lead (21) from the voltage source (20) to the positive electrode (18) or syringe needle (14) and connect the negative lead (22) from the voltage source (20) to the negative electrode (19). Verify that the voltage source (20) delivers about −1500 V.

The EHD-NP (4) produced by mixing the OP with the AP under influence of the electrical field can be concentrated using centrifugal filtration. The contents of the 20 mL glass vial (17) can be transferred to a 100 kDa cutoff centrifugal ultrafiltration column (“CUC”), as an example, SigmaAldrich PN UFC9010D Amicon® Ultra-15 Centrifugal Filter Unit. Centrifuge the CUC at 3000 RCF for 30 min. at about 25° C. (about 77° F.). Transfer 10 mL 50 mM sodium borate, 100 mM sodium phosphate, 7.3-7.5 pH (“borate buffer”) to the CUC. Centrifuge the CUC at 3000 RCF for 30 min. at about 25° C. (about 77° F.). Transfer EHD-NP filtrate from the CUC into a 1.5 mL microcentrifuge tube. Measure and record the volume of the collected EHD-NP filtrate. Transfer 15 μl of EHD-NP filtrate from the 1.5 mL microcentrifuge tube to a 1.5 mL tube and add 285 μl borate buffer. Transfer 290 μl of EHD-NP filtrate to a spectrophotometer cuvette. Measure and record optical density with a spectrophotometer at 450 nm (OD₄₅₀).

The EHD-NP filtrate may contain aggregates of IsoDots (4a) or aggregates of polymer (P) (3) lacking a ID (2a) (individually and collectively “aggregate”). The aggregate can be substantially removed from the EHD-NP filtrate to produce substantially pure IsoDots (4a). A scalable method for purification of IsoDots (4a) can include one or more of: transfer of 120 μl aliquots of the EHD-NP filtrate from the centrifugal ultrafiltration column to corresponding 1.5 mL tubes. Centrifuge the 1.5 mL tubes containing the EHD-NP filtrate at 3,000 RCF for 10 min. to pellet aggregates. Without disturbing the aggregate pellet, remove IsoDots (4a) containing supernatant. Measure and record the volume of the IsoDots supernatant. Mix 15 μl of IsoDots supernatant and 285 μl of borate buffer in 1.5 mL tube. Transfer 290 μl of the mixture to a spectrophotometer cuvette. Measure and record optical density with spectrophotometer at 450 nm (OD₄₅₀).

Embodiments of the PNEP (4)(IsoDots (4a), MagDots (4b), QDots (4c) and/or MultiDots (4d)) evidence hydrodynamic diameter (“HD”), polydispersity index (“PDI”) and size stability suitable for effective clinical and non-clinical applications. Hydrodynamic diameter and PDI can be obtained by performing dynamic light scattering measurements using NanoBrook 90 Plus particle size analyzer. Size histograms can be plotted using SigmaPlot (Systat Software Inc., San Jose, CA, U.S.A.), and size distributions can be fit to log-normal distributions.

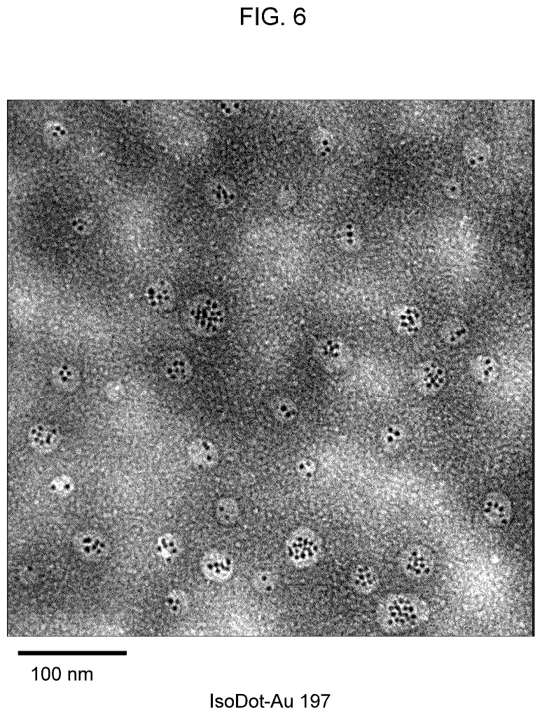

HD is defined as the size of a hypothetical hard sphere that diffuses in the same fashion as that of the particle being measured. Though in practice, macromolecules or particles in solution are solvated, dynamic and non-spherical. Due to this, the diameter calculated from the particle's diffusional properties will signify the apparent size of the solvated/dynamic hydrated particle. The HD of the PENP (4) produced by the above EHD method can vary depending on the parameters used during EHD to produce PENP (4). The TEM micrographs shown in FIGS. 6 through 23 evidence PENP (4) having substantially consistent HD of about 20 nm to about 500 nm; however, by varying the parameters used during EHD, PENP-IsoDots (4a) HD can vary in a range of about 40 nm to about 100 nm.

PDI is a representation of the distribution of size populations within a particle sample. The numerical value of PDI ranges from 0.0 for a perfectly uniform population within a particle sample to 1.0 for a highly polydisperse population within a particle sample. PDI values of 0.2 and below are deemed acceptable in practice for polymer-based nanoparticle materials. The calculations used for the determination of PDI are defined in the ISO standard documents 13321:1996 E and ISO 22412:2008. IsoDots (4a) can have a substantially consistent PDI of about 0.1 to about 0.2.

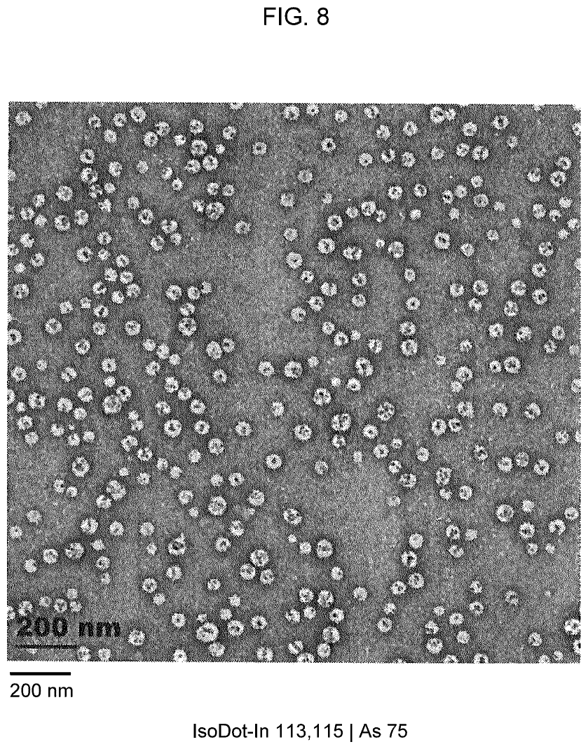

Now, with primary reference to FIGS. 6A through 9, TEM micrographs evidence that IsoDots (4a) can be formed to encapsulate MI (2a) having a range of atomic mass in the range of 187 amu to 239.26 amu, including, but not necessarily limited to, the metal isotopes described in the forging list of isotopes, while retaining a substantially uniform and narrow size distribution. In particular embodiments, the IsoDots (4a) can be loaded with MI (2a) having atomic mass suitable for analysis by current generation mass cytometers in the range of 75 amu to 209 amu. The illustrative examples of FIGS. 6 through 9, evidence IsoDots (4a) formed to encapsulate: gold-197 (“Au197”) (as shown in the example of FIG. 6), lead sulfide (PbS) having a mass of 239.26 amu (PbS 239.26) (as shown the example of FIGS. 7A and 7B), indium 113, 115/arsenic 75 (In113, 115/As 75) (as shown in the example of FIG. 8), and cadmium-106, 114/telluride-130 (Cd 106, 114/Te130) (as shown the example of FIGS. 9A and 9B).

Embodiments of the PENP (4)(4a, 4b, 4c, 4d) address long felt but unresolved concerns in regard to the lack of available nanoparticle mass tags in a nanocomposite having uniform and narrow size distribution in the range of about 20 nm to about 100 nm which allow efficient specific physical adsorption through cell membranes. Moreover, PENP (4) load a greater number of IP (2a), SPION (2b), MI (2c) and QD (2d) as compared to conventional chelating polymers, which by way of the increased load allows for a lower limit of detection in mass cytometry.

### MagDots.

Now, with primary reference to FIG. 4B and FIG. 5, superparamagnetic iron oxide nanoparticles (“SPION”) (2b) are small synthetic particles of maghemite having an empirical formula of Fe₂O₃, γ—Fe₂O₃ and a molar mass 159.69 g/mol (CAS No. 1309-37-1) or magnetite having an empirical formula of Fe₃O₄ and a molar mass of 231.53 g/mol (CAS NO. 1317-61-9), or combinations thereof, with a core ranging from about 5 nm to about 30 nm in diameter. Magnetite and maghemite nanoparticles are the most widely used SPION (2b) in various biomedical applications. SPION (2b) exhibit the phenomenon of “superparamagnetism.” The particles that exhibit superparamagnetism, on application of an external magnetic field (15) (as shown in the example of FIG. 5), become magnetized up to their saturation magnetization, and on removal of the magnetic field, no longer exhibit any residual magnetic interaction. This property is size-dependent and generally arises when the size of nanoparticles reaches about 5 nm. At such a small size, these nanoparticles do not exhibit multiple domains as found in large magnets; on the other hand, they become a single magnetic domain and act as a “single super spin” that exhibits high magnetic susceptibility. Thus, on application of a magnetic field, these nanoparticles provide a stronger and more rapid magnetic response compared with bulk magnets with negligible remanence (residual magnetization) and coercivity (the field required to bring the magnetism to zero). Once the applied magnetic field is removed, the magnetic particles retain no residual magnetism at room temperature and are easily dispersed. Uncoated iron oxide NPs have very low solubility that can lead to precipitation due to gravitation forces and also a high rate of agglomeration under physiological conditions. Thus, to be used effectively, SPION can be capped with an amphiphilic coating.

In particular embodiments, SPION (2b) capped with a ligand (16), such as oleic acid, can have particle sizes in the range of about 5 nm to about 30 nm. Various SPION (2b) can be obtained from Ocean NanoTech, PN SOR05 to SOR30. Illustrative examples of embodiments, include SPION (2b) capped with oleic acid having particle sizes of 5 nm, 10 nm, 15 nm, and 20 nm. However, these illustrative SPION (2b) particle sizes are not intended to preclude the use of SPION (2b) having lesser or greater particle size, or combinations of SPION (2b) particle sizes, which can be used alone or in combination with one or more QD (2a) in embodiments of the PENP (4).

Typically, capped SPION (2b) in accordance with the invention reach a required level of water solubility and biocompatibility by surrounding the SPION (2b), or SPION/IP (2a, 2b) combination(s), with a polymer (3). A polymer (3) encapsulation layer suitable for use with embodiments of the invention include PS-b-PEG-NH₂ obtained in FIG. 2A, Block 2I or FIG. 2B, Block 2D of the synthesis process, above described. Embodiments of the invention can be produced through the use of various combinations of one or more of: SPION (2b) or SPION/QD (2b, 2d), capping ligand (16), and PS-b-PEG-NH₂ having PS molecular weights ranging from about 1.5 kDa to about 40 kDa and PEG having molecular weights ranging from about 10 kDa to about 40 kDa.

An illustrative example of a scalable method of production MagDots (4b) by EHD can include one or more of: PS-b-PEG-NH₂ obtained in FIG. 2A, Block 2I or FIG. 2B, Block 2D at 10 mg/mL into a first 1.5 mL tube. Introduce 13 μl of the desired SPION (2b) particle from 25 mg/mL in toluene (e.g. 20 nm) into a second 1.5 mL tube. Transfer 52 μl of acetone/methanol (60/40) to the SPION in the second 1.5 mL tube. Mix by shaking. Centrifuge the second 1.5 mL tube containing the mixed SPION (2b) at 7000 RPM for 1 min. at about 25° C. (about 77° F.). Immediately remove supernatant with a 200 μl pipette. Immediately, transfer 13 μl of anhydrous tetrahydrofuran (“THF”) to the second 1.5 mL tube containing the SPION (2b) and mix thoroughly with a pipette. Transfer 65 μl of a QD (2a) (from 5 mg/mL in toluene) into third 1.5 mL micro centrifuge tube. Transfer 130 μl of acetone/methanol (60/40 v/v) to the third 1.5 mL microcentrifuge tube containing the QD. Tilt to gently mix the QD (2a). Centrifuge the third 1.5 mL microcentrifuge tube containing QD at 7000 R.P.M. for 1 minute at 25° C. (about 77° F.). Remove the supernatant with 200 μl pipette. Transfer 65 μl of THF to the third 1.5 mL microcentrifuge tube. Mix thoroughly with an air displacement pipette. To a 4 mL vial, add 322 μL of THF. Transfer 13 μL of the SPION (2b) in THE contained in the second 1.5 μL microcentrifuge tube into the 4 mL vial. Optionally, transfer 65 μL of the MI (2a) in THE contained in the third 1.5 mL microcentrifuge tube into the 4 mL vial. Transfer 100 μL of the PS-b-PEG-NH₂ into the 4 mL vial to produce the OP for EHD.

Particular embodiments, EHD can be performed by cleaning the EHD mixing system syringe (11) three times with THF. Load about 0.5 mL of the OP into the syringe barrel (12). Mix the inorganics in the OP thoroughly while loading the syringe (11). Attach the syringe (11) to the syringe pump (15) and set the syringe pump (15) to generate a flow rate of the OP containing the inorganics from the syringe needle (14) in the range of about 11.00 mL/hr. to about 14.00 mL/hr. In particular embodiment the flow rate can be about 12.5 mL/hr. Prime the syringe needle (14) until a drop of the OP forms at the end of the syringe needle (14). Twice rinse a 20 mL glass vial (17) with distilled or deionized water (individually or collectively “DI water”). Introduce about 10 mL of DI water into the 20 mL glass vial (17). Submerge the syringe needle (14) into the 20 mL glass vial (17). Clean the negative electrode (19) by submerging in THF, wipe, and rinse with DI water. Place the negative electrode (19) into the AP contained in the 20 mL glass vial (17). Place the positive electrode (18) into the AP contained in the 20 mL glass vial (17). In particular embodiments the syringe needle (14), if electrically conductive, can act as the positive electrode (18). Observe that the positive electrode (18), the negative electrode (19), and the syringe needle (14) do not contact. Connect the positive lead (21) from the voltage source (20) to the positive electrode (18) or syringe needle (14) and connect the negative lead (22) from the voltage source (20) to the negative electrode (19). Verify that the power source delivers about −1000 V.

The EHD-PENP (4) produced by mixing the organic phase mixed with the DI water under influence of the electrical field can be concentrated using centrifugal filtration. The contents of the 20 mL glass vial (17) can be transferred to a 100 kDa cutoff centrifugal ultrafiltration column available from SigmaAldrich, PN UFC9010D Amicon® Ultra-15 Centrifugal Filter Unit. Centrifuge at 2000 RCF for 30 min. at about 25° C. (about 77° F.). Transfer EHD-PENP (4) from the centrifugal ultrafiltration column to a 1.5 mL microcentrifuge tube. Measure and record the EHD-NP volume. Transfer 5 μL of EHD-PENP (4) filtrate from the 1.5 mL microcentrifuge tube to a 1.5 mL tube and add 295 μl borate buffer. Transfer 290 μl of EHD-PENP (4) to a spectrophotometer cuvette. Measure and record optical density with spectrophotometer at 450 nm (OD₄₅₀).

The EHD-PENP filtrate may contain aggregates of PENP (4) or aggregates of PENP lacking a SPION (Sb) or a QD (2a) (collectively “aggregate”). The aggregates can be substantially removed from the EHD-NP filtrate to produce substantially pure MagDots (4b). As one example, a scalable size exclusion process to purify MagDots (4b) can include: place a magnetic particle separation column (“MPSC”) in a magnetic field gradient (e.g. up to about 100-200 T/m). Transfer 300 μl of borate buffer into MPSC. Allow the borate buffer to pass through the MPSC. Introduce the EHD-NP filtrate into the MPSC in the magnetic field (23). Allow the filtrate liquid to pass through the MPSC. Transfer 300 μl of borate buffer into MPSC. Mix the EHD-NP in the borate buffer in the MPSC. Allow all of the borate buffer to pass through the MPSC. Extract the MPSC from the magnetic field gradient. Once the MPSC is extracted from the magnetic field gradient (23) the MPSC can no longer retain the MagDots (4b) which can then be eluted. Remove any liquid from MPSC tip. Place the MPSC in a MagDots (4b) collection tube labeled MagDots-Wavelength. Transfer 200 μl of borate buffer into the MPSC. Pipette to mix MagDots (4b) in the borate buffer in the MPSC. Collect MagDots (4b) into the MagDots-Wavelength collection tube. Mix collected MagDots (4b) thoroughly with an air displacement pipette. Measure and record the volume of the collected MagDots (4b). Combine 5 μl of MagDots (4b) and 295 μl of borate buffer. Transfer 290 μl of sample to spectrophotometer cuvette. Measure and record optical density with spectrophotometer at 450 nm (OD₄₅₀).

Now, with primary reference to FIGS. 10 through 12 including TEM micrographs illustrating MagDots (4b) produced by the above method loaded with SPION (2b). The morphology of MagDots (4b) produced by the above EHD-EM-NP method can be characterized by the use of TEM image and dynamic light scattering of the MagDots (4b) to determine HD and PDI, as above described. MagDots (4b) can be formed to encapsule SPION (2b) having a particle size of 20 nm (as shown in the example of FIG. 10), 15 nm (as shown in the example of 11) and 5 nm (as shown in the example of FIG. 12) respectively.

DLS can also be used in determining the size of MagDots (4b). During DLS measurement, a suspension of MagDots (4b) can be exposed to a light beam and as the incident light impinges on the MagDots (4b), the direction and intensity of the light beam can be altered due to scattering. MagDots (4b) in suspension are in constant random motion due to their kinetic energy, the variation of the intensity with time, therefore, contains information on that random motion and can be used to measure the diffusion coefficient of the particles. The HD of the MagDots RH can be calculated from its diffusion coefficient by the Stokes-Einstein equation D_{f}=k_{B}T/6πηR_{H}, where k_{B} is the Boltzmann constant, T is the temperature of the suspension, and η is the viscosity of the surrounding media.

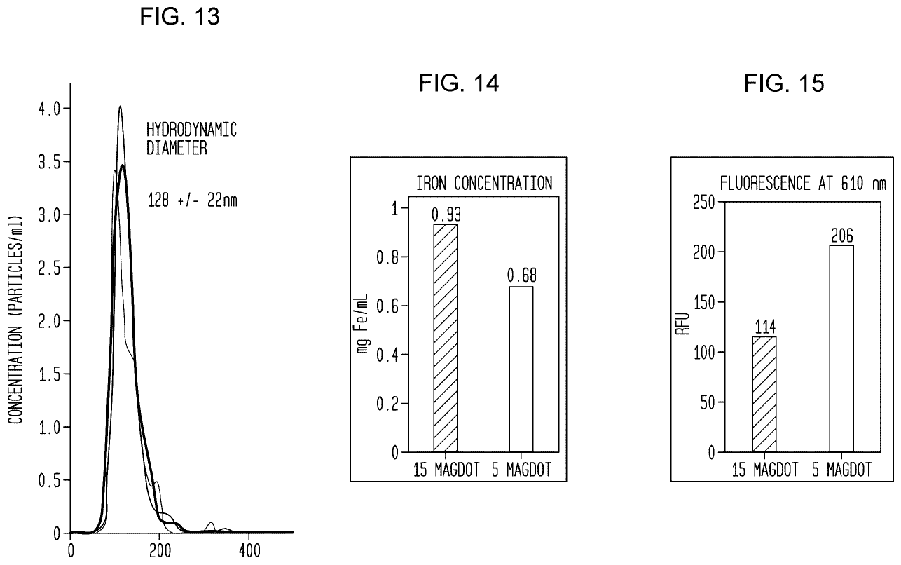

Now, with primary reference to FIG. 13, nanoparticle tracking analysis (NTA) of each of the MagDots15 shown in the example of FIG. 11 can be illustrated as a plot of particle size versus particle concentration to evidence that the HD of each population can have a uniform size with a PDI of about 0.1 to about 0.2. The determined HD of the MagDots15 can be about 120 nm to about 140 nm and the determined HD of the MagDots5 can be about 180 nm to about 220 nm.

Based on the TEM analysis or DLS or NTA analysis, the HD of the MagDots (4b) produced by the above EHD method can vary depending on the parameters used during EHD and occur within a range of about 40 nm to about 500 nm, and evidence that MagDots (4b) populations evidence substantially uniform HD and a PDI of about 0.1 to about 0.2.

Now, with primary reference to FIG. 14 comprising a bar graph comparing the iron concentration of MagDots 5 and MagDots 15, evidence that MagDots 15 can be produced having a greater iron concentration than MagDots 5.

Now, with primary reference to FIG. 15 comprising a bar graph comparing the fluorescence of MagDots 5 and MagDots 15, evidence that MagDots 5 can be produced having a greater fluorescence than MagDots15.

Thus, by pre-selection of one or more of: SPION (2b) size, MI (2a), PS-b-PEG-NH₂ (3) which can include PS molecular weights ranging from about 1.5 kDa to about 40 kDa and PEG having molecular weights ranging from about 10 kDa to about 40 kDa, and parameters of EHD, and the ratio of MI:SPION:polymer, numerous and varied embodiments of the MagDots (4b) can be produced having utility in a correspondingly numerous clinical and non-clinical applications.

### Preparation of Antibodies.

Again, with primary reference to FIG. 1, the polymer nanomaterial encapsulation system (1) can further include the preparation of antibodies (Ab) (5). As used herein, an “antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. Antibodies can be polyclonal or monoclonal, derived from serum, a hybridoma or recombinantly cloned, and can also be chimeric, primatized, or humanized.

In particular embodiments, the PENP (4) produced by FNP, EHD, EM-NPa, self-assembly, or by other means, can be targets for site directed conjugation to antibodies (Ab) (5′). As one illustrative example, half-antibodies can be produced by preferential reduction of the disulfide bonds in the antibody hinge region to yield monovalent components with free thiol groups (“SH”) that can be employed for site-directed conjugation to PENP (4), IsoDots (4a), MagDots (4b), QDots (4c), MultiDots (4d) or combinations thereof. The reduced antibody fragments can be prepared by reacting 2-mercaptoethylamine hydrochloride (2-MEA), dithiotreitol, mercaptoethanol, or tris(2-carboxyethyl)phosphine (TCEP), to produce a reduced half-antibody as shown in Formula XII.

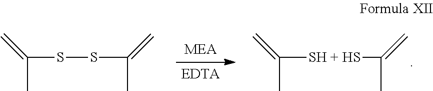

However, this example is not intended to preclude embodiments that may include one or more of: full antibody molecules or their F(ab′)2, Fab′, Fv, rIgF, Fc fragments obtained after pepsin or other enzymatic digestion, or combinations thereof.

A particular illustrative example of a scalable process to produce half-antibodies useful for conjugation to PENP (4), IsoDots (4a), MagDots (4b), QDots (4c) and MultiDots (4d) can include: preparing an antibody stock solution including Immunoglobulin G (“IgG”) (0.5 mg/mL) in a buffer solution of 100 mM phosphate buffer saline (“PBS”) with 10 mM ethylenediaminetetraacetic acid disodium salt dihydrate (“EDTA”) at pH 7.4 (“PBS buffer solution”). An aliquot of 160 μl of the IgG stock solution can be transferred to a first 1.5 mL tube. 2-MEA·HCl having a linear formula of HSCH₂CH₂NH₂·HCl and a molecular weight of 113.60 (CAS NO: 156-57-0) can be obtained from Fisher Scientific PN AAA1437714. In a second 1.5 mL tube, 15 mg of 2-MEA·HCl can be dissolved in 264 μl PBS buffer solution by pipetting. Transfer 5.0 μl of the 2-MEA·HCL solution, respectively, to the 160 μl of the IgG stock solution in the first 1.5 mL tube, vortex, and place 1.5 mL tube containing the IgG solution in a water bath at 37° C. (about 98.6° F.) for 90 min.

The resulting reduced antibody product (also, referred to as “half-antibody product”) (5) may require desalting and a buffer exchange. Desalting removes unreacted MEA contaminants from the half-antibody product while exchanging the half-antibodies into reaction buffer for conjugation to the PENP (4), IsoDots (4a), and/or MagDots (4b). An illustrative example of a scalable procedure for desalting and buffer exchange can include utilization of ZEBA™ spin desalting columns obtained from ThermoFisher Scientific, PN 89883, or equivalents thereof. A pair of desalting columns can each be prepared by removing the bottom closure and placing the columns in a 1.5 mL collection tube. Centrifuge at 1500×g for 1 min. to remove storage solution from the desalting column resin. Add 300 μl of 1×100 mM Sodium Phosphate, 1 mM EDTA in water at pH: 6.95-7 (rinse buffer”) or appropriate buffer to the top of the resin bed and centrifuge at 1500 RFC for 1 min. Discard flow-through from collection tube. Repeat addition of 300 μl of 1× rinse buffer two additional times, discarding buffer from the collection tube each time.

The desalting column can now be loaded with the reduced antibody (5) product by placing the first equilibrated desalting column into a first 1.5 mL collection tube marked reduced antibody 1 (“R-Ab-1”), remove the cap from the top of the first desalting column and slowly apply up to a 160 μl reduced antibody product to the center of the compact resin bed. For sample volumes less than 70 μl, apply 15 μl buffer (stacker) to the top of the resin bed after the sample has fully absorbed to ensure maximal protein recovery. Centrifuge at 1500 RCF for 2 min. at 15° C. to 20° C. (59° F. to 68° F.) to collect desalted reduced antibody product. Place the second equilibrated desalting column into a second 1.5 mL collection tube marked reduced antibody 1 (“R-Ab-2”), remove the cap from the top of the second equilibrated desalting column. Transfer the collected reduced antibody (5) product from collection tube R-Ab-1, to the top of the second desalting column. Centrifuge at 1500 RCF for 2 min. at 15° C. to 20° C. (59° F. to 68° F.) to collect desalted reduced antibody product in collection tube R-Ab-2. Cap collection tube R-Ab-2 and place at RT.

### Preparation of Half-Antibody-Polymer Nanocomposite Conjugates.

Now, with primary reference to FIG. 1, the polymer nanomaterial encapsulation system (1) can further include the surface functionalization of PENP (4), IsoDots (4a), MagDots (4b), QDots (4c) or MultiDots (4d) by addition of the reduced antibody product FIG. 1, Block 1 D. As a basis for modification, chemical groups such as amines, carboxylates, thiols, or other reactive groups can be introduced into the PENP (4), IsoDots (4a), MagDots (4b), QDots (4c) or MultiDots (4d) during synthesis, as above described. These groups can be targeted by heterobifunctional chemical cross-linkers (10) including reactive chemical groups connected by spacer arms (as examples: alkane, polyethylene glycol, cleavable disulfide spacers) having various lengths and functions, imparting flexibility, solubility, or other desirable characteristics to the functionalized PENP (4), MultiDots (4a), MagDots (4b), QDots (4c) or MultiDots (4d). As examples, PENP functional group(s) (9) can be reacted with the first reactive group (10′) of the heterobifunctional chemical cross-linkers (10). As illustrative examples, where the functional group (9) of the PENP (4), IsoDots (4a), MagDots (4b), QDots (4c) or MultiDots (4d) comprises an amine, the amine can be reacted with a succinimidyl esters to form amide bonds, or comprises a carboxylate, the carboxylate can be reacted with carbodiimides to produce O-acylisourea intermediates that can be reacted with amines to form amide bonds, or comprise a thiol, the thiol can be reacted with maleimide to form thioether bonds. The reduced antibody (5) product can be reacted with the second reactive group (10″) of the heterobifunctional chemical cross-linker (as shown in the example of FIG. 4C). For example, the second reactive group (10″) of the heterobifunctional chemical cross-linker (10) can comprise a maleimide which can react with the sulfhydryl of the half anti-body (5) to form a thioether bond.

In particular embodiments, both the reactive group associated with the PENP (4) can be modified by reaction with the first reactive group (10a′) of a first heterobifunctional cross-linker (10a) and the reduced antibody (5) product can modified by reaction with the first reactive group (10b′) of a second heterobifunctional chemical cross-linker (10b). The first and second chemical cross-linkers (10a, 10b) can be selected to include second reactive groups (10a″, 10b″) that remain stable in physiological aqueous buffers and upon admixture proceed with fast kinetics and high selectivity to create a covalent bond.

In particular embodiments, the reduced antibody (5) product in collection tube R-Ab-2 can be modified for subsequent conjugation to PENP (4) by reaction of the free sulfhydryl group of the half-antibody (5) with a first reactive group (10a′) comprising a maleimide of a first heterobifunctional cross-linker (10a) to form a thioether bond. In an illustrative example, the first heterobifunctional cross-linker (10a) can comprise sulfo trans-cyclooctene maleimide (“Sulfo-TCO Maleimide”) having a linear formula C₁₇H₂₇N₃O₈S (CAS No. n/a) and a molecular weight of 457.50 g/mol can be obtained from Click Chemistry Tools, PN 1355 comprising Formula XIII.

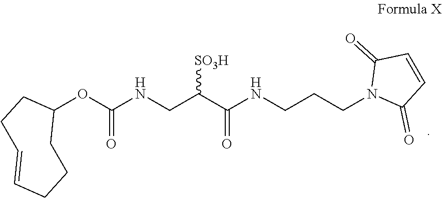 [SMILES: O=C(NCC(C(=O)NCCCN1C(=O)C=CC1=O)S(=O)(=O)O)OC1CC/C=C/CCC1]

Sulfo TCO-Maleimide comprises a water-soluble reagent that enables incorporation of the TCO moiety onto thiol-containing half-antibody. The maleimide group specifically reacts with the sulfhydryl group (“SH”) at pH 6.5 to 7.5 to form a stable thioether bond. The hydrophilic sulfonated spacer arm greatly improves water solubility of Sulfo TCO antibody (“Sulfo-TCO-Ab”) comprising the Formula XIV.

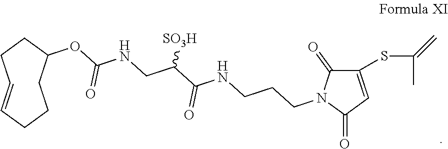 [SMILES: C=C(C)SC1=CC(=O)N(CCCNC(=O)C(CNC(=O)OC2CC/C=C/CCC2)S(=O)(=O)O)C1=O]

An illustrative example of a scalable process to produce the Sulfo-TCO-Ab useful for conjugation to PENP can include one or more of: remove a tube containing 0.1 mg Sulfo-TCO-maleimide from −20° C. (−4° F.) and bring to RT over 15 min. Add 46.4 μl of anhydrous dimethysufoxide (“DMSO”) having a linear formula C₂H₆OS and a molecular weight of 78.13 g/mol. (CAS No. 67-68-5) to 0.1 mg Sulfo-TCO-maleimide. Vortex the Sulfo-TCO-maleimide in the tube for 10 min. to ensure dissolution in the DMSO. Repeatedly mix the Sulfo-TCO-maleimide in DMSO via pipette set to 60 μl. Add 5 μl or 10 μl of Sulfo-TCO-maleimide to the R-Ab-2 tube containing the reduced antibody (5). Discard the remaining Sulfo-TCO-maleimide. Relabel the tube containing the Sulfo-TCO-Ab. Cap and vortex the tube containing the Sulfo-TCO-Ab. Place the Sulfo-TCO-Ab tube at 4° C. (39.2° F.) for 20-24 hr.

The PENP (4) products above described can be reacted with a first reactive group of a second heterobifunctional cross-linker. The modified PENP (4) can then be associated with one or more target moieties (24). The term “target moiety” as used herein means any molecule found inside an organism, for example, a specific cell, a cell organelle, a protein, a peptide, an amino a acid nucleic acid, an oligonucleotide, a carbohydrate, a lipid, a metabolite, by selective reaction of the second reactive group with the corresponding function groups within the target moiety (24). Illustrative embodiments of the nanocomposite antibody conjugate include one or more of anti-CD3, CD4, CD8, CD16, or anti-CD32 antibodies capable of specifically binding CD3, CD4, CD 8, CD16 and or CD32 T cells.

In particular embodiments, the second heterobifunctional cross-linker (10b) can comprise methyltetrazine polyethylene glycol-4-N-hydroxysuccinimide ester (“TZ-PEG₄-NHS ester”) having a linear formula C₂₄H₃₁N₅O₉ having a molecular weight of 533.53 g/mol (CAS NO. 182907-92-1) comprising Formula XVI.

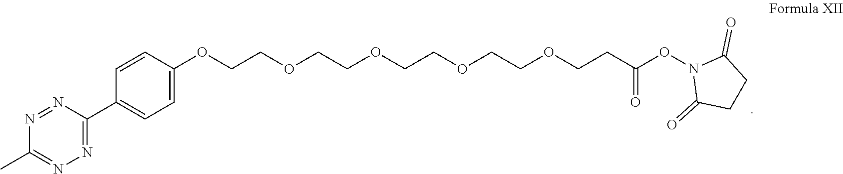 [SMILES: Cc1nnc(-c2ccc(OCCOCCOCCOCCOCCC(=O)ON3C(=O)CCC3=O)cc2)nn1]

An illustrative example, the TZ-PEG4-NHS ester comprising Formula XIII can be reacted with the functional group (9) of the multi-arm PEG-NH₂ (3) comprising multiple amine as shown in Formula IV or Formula V of the PENP (4) to produce PENP-PEG₄-TZ comprising Formula XVII.

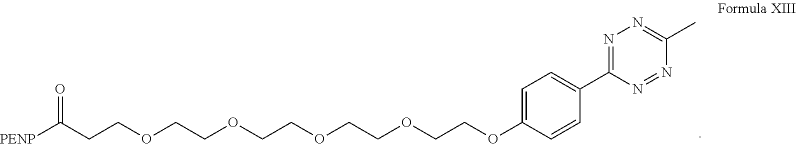

As one example, a scalable process to produce the PENP-PEG₄-TZ of Formula XIII using the PENP (4) functional group (9) comprising an amine of the multi-arm PEG-NH₂ (3) shown in Formula IV or Formula V can include one or more of: bring 1 mg of TZ-PEG-NHS ester to RT over approximately 15 min. Transfer 93.7 μl or 375 μl of anhydrous DMSO to TZ-PEG4-NHS ester to produce 20 mM or 5 mM TZ-PEG4-NHS in DMSO. Mix TZ-PEG4-NHS ester in DMSO by pipette for complete dissolution of TZ-PEG4-NHS ester in DMSO. Label 1.5 mL tube as PENP-Wavelength-TZ. Transfer PENP into PENP-Wavelength-TZ tube (120 μl of MagDots (4b) or 80 μl of IsoDots (4a)). In IsoDots (4a) embodiments about 30 nmol of amine can be activated by reaction with about 70 nmol of TZ-PEG4-NHS ester and in MagDot (4b) embodiments about 120 nmol of amine can be activated by reaction with 70 nmol of TZ-PEG4-NHS ester. Pipette 3.5 μL of 20 mM TZ-PEG4-NHS ester into the IsoDot-Wavelength-TZ tube or 15 μl of 5 mM TZ-PEG4-NHS ester into the MagDot-Wavelength-TZ tube. Mix thoroughly with an air displacement pipette. React at RT for 2 hrs.

In particular embodiments the PENP-PEG4-TZ comprising Formula XVIII can be reacted with Sulfo-TCO-Ab comprising the Formula XI in an inverse-electron demand Diels-Alder [4+2] cycloaddition reaction of TCO with TZ to form a dihydropyridazine bond to produce conjugate PENP-Antibodies (6) (“PENP-Ab”) comprising Formula XIV including but not limited to IsoDots (4a) shown in the example of FIG. 4A or MagDots (4b) as shown in the example of FIG. 4B, where R₁ comprises PENP and where R₂ comprises an Ab.

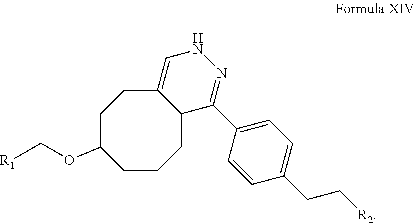

As one example, a scalable process to produce the PENP-Ab (6) of Formula XIV using PENP-PEG4-TZ (MagDots-PEG4-TZ or IsoDots-PEG4-TZ) and Sulfo-TCO-Ab can include a pair of desalting columns (as a non-limiting example Zeba™ desalting columns) each prepared by removing the bottom closure and placing each of the pair of desalting columns in a 1.5 mL collection tube. Centrifuge at 1500×g from 1 min. to remove storage solution from the desalting column resin. Add 300 μL of 1× rinse buffer or appropriate buffer to the top of the resin bed and centrifuge at 1500 RFC for 1 minute. Discard flow-through from the respective collection tubes. Repeat addition of 300 μL of 1× rinse buffer (pH 6.0) two additional times, discarding buffer from the respective collection tubes each time. Transfer the PENP-PEG4-TZ to the first of the pair of desalting columns. Transfer the Sulfo-TCO-Ab to the second of the pair of desalting columns. Centrifuge the first and second desalting columns at 1500 RCF for 2 min. at 15° C. to 20° C. (59° F. to 68° F.) to collect PENP-PEG4-TZ and Sulfo-TCO-Ab products post excess unconjugated linker removal. Mix the desalted PENP-PEG4-TZ (120 μL MagDots or 80 μL IsoDot) in the first collection tube by pipette. Transfer the desalted 160 μL (80 μg) Sulfo-TCO-Ab from the second collection tube into the PENP-PEG4-TZ in the first collection tube. Let react for 24 hrs. at 4° C. (39.2° F.) to produce PENP-Ab (MagDots-Ab (6b) or IsoDots-Ab (6a)).

Subsequent to conjugation, the resulting PENP-Ab (6) product can be purified. As one example, a scalable size exclusion process to purify IsoDots-Ab (6a) can include: Addition of Captocore C700 (Cytvia 17548102) or Captocore C400 (Cytvia 17372402) resin to a chromatography column and wash the column with PBS buffer (0.1 M sodium phosphate, 0.15 M NaCl buffer at pH 7.2). Add the IsoDot-Ab (6a) solution to the Captocore Resin. Allow the IsoDot-Ab (6a) solution to incubate and mix with the resin for 15 minutes at 1000 rpm. Centrifuge the column and retrieve the IsoDot-Ab (6a) solution free of antibody.

A scalable size exclusion process to purify MagDots-Ab (6b) can include: mix MagDots-Ab (6b) in conjugation buffer thoroughly with an air displacement pipette. Place a MPSC in a magnetic field gradient (e.g. up to about 100-200 T/m). Introduce the MagDots-Ab (6b) in conjugation buffer into the MPSC. Allow the conjugation buffer to pass through the MPSC. Transfer 300 μL of BBS into MPSC. Pipette to mix MagDots-Ab (6b) in reaction buffer in the MPSC. Allow the reaction buffer to pass through the MPSC. Transfer 300 μL of reaction buffer into the MPSC. Pipette to mix MagDots-Ab (6b) in reaction buffer in the MPSC. Allow the reaction buffer to pass through the MPSC. Remove any reaction buffer from tip of the MPSC. Extract the MPSC from the magnetic field gradient. Once the MPSC is extracted from the magnetic field gradient, the MPSC can no longer retain the MagDots-Ab (6b) and the MagDots-Ab (6b) can be eluted. Place the MPSC in a MagDots-Ab (6b) collection tube labeled MagDots-Ab. Transfer 200 μl of reaction buffer into the MPSC. Pipette to mix MagDots-Ab (6b) in reaction buffer in the MPSC. Collect MagDots-Ab (6b) into the MagDot-Antibody-Date collection tube. Mix collected MagDots-Ab (6b) thoroughly with an air displacement pipette.

### Cell Labelling Utilizing IsoDots-Ab-Cellular Target.

As one illustrative example of IsoDots-Ab (6a) labeling of cellular targets (24′), peripheral blood mononuclear cells (“PBMC”) (24′) can be labeled using IsoDots-Ab (6a). A scalable method of labeling PBMC with IsoDots-Ab (6a) can include one or more of: diluting peripheral blood as an iso-osmatic solution 50:50 with Hank's balanced salt solution (“HBSS”) (SigmaAldrich, PN H6648). Diluted peripheral blood can be layered over a Ficoll-Hypaque gradient (density=1.077 g/cm³) and centrifuged at 1350 RPM for 30 min. without brake. Serum can be aspirated and discarded. PBMC can be removed and transferred to another collection tube and washed with PBS. The PBMC pellet can be resuspended in 10 mL PBS and cell counts performed on a hemacytometer. A million PBMC (24′) in 200 μl of PBS can be placed in a 12×75 mm flow cytometry tube to which 10 μl of IsoDots-Ab mouse anti human CD8 (6a) was added. PBMC (24′) and IsoDots-Ab mouse anti human CD8 (6a) incubated for 25 min. at RT. After expiration of the incubation period, 500 μl of additional PBS can be added to the IsoDots-Ab mouse anti human CD8 bound PBMC (7) (“IsoDots-Ab mouse anti human CD8-PBMC”). The IsoDots-Ab mouse anti human CD8-PBMC (7) were centrifuged for 7 min. at 1800 RPM at RT.

The supernatant can be discarded and the PBMC/IsoDots-Ab mouse anti human CD8-PBMC (7) can be re-suspended in 400 μl of PBS for flow cytometry analysis (8) for detection of IsoDots-Ab mouse anti human CD8-PBMC (7) population using a Cytek Northern Lights spectral flow cytometer. The CD8 protein complex can be an important T cell marker for the classification of malignant lymphomas and leukemias (T cell neoplasms). CD8 can also be used for the identification of T cells in coeliac disease, lymphocytic colitis and collagenous colitis. IsoDots-Ab mouse anti human CD3, CD4, CD8, CD16, or CD32-PBMC can be isolated as described above.

### Flow Cytometry of IsoDots-Ab-Cellular Target.

IsoDots-Ab (6a) and/or MagDots-Ab (6b) can effectively and specifically label target moieties (24) comprising cellular targets (24′). Single particle imaging, cellular imaging, or flow cytometry using IsoDots-Ab (6a) tagged cellular targets (24′) evidence that IsoDots-Ab (6a) can effectively and specifically label cell surface receptors and subcellular structures in both live or fixed cells without any detectable non-specific binding. Flow cytometry can be performed to evaluate IsoDots-Ab (6a) performance and to show CD 45 labeling as compared to conventional cell labeling.

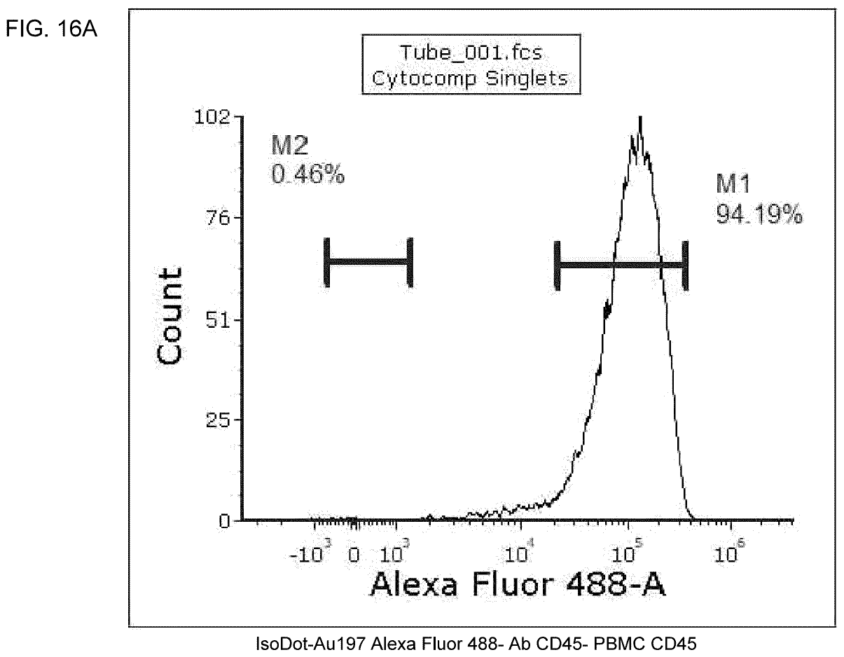

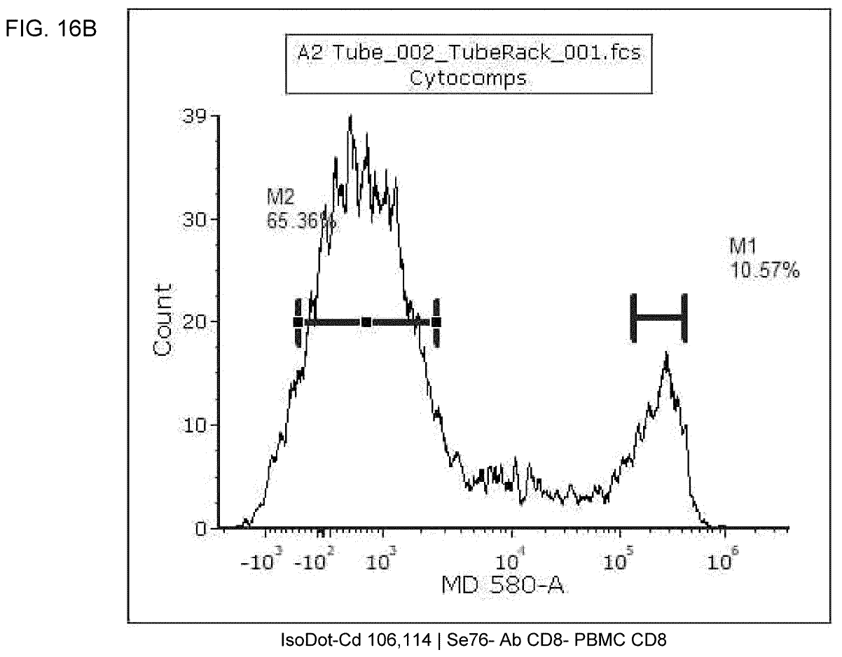

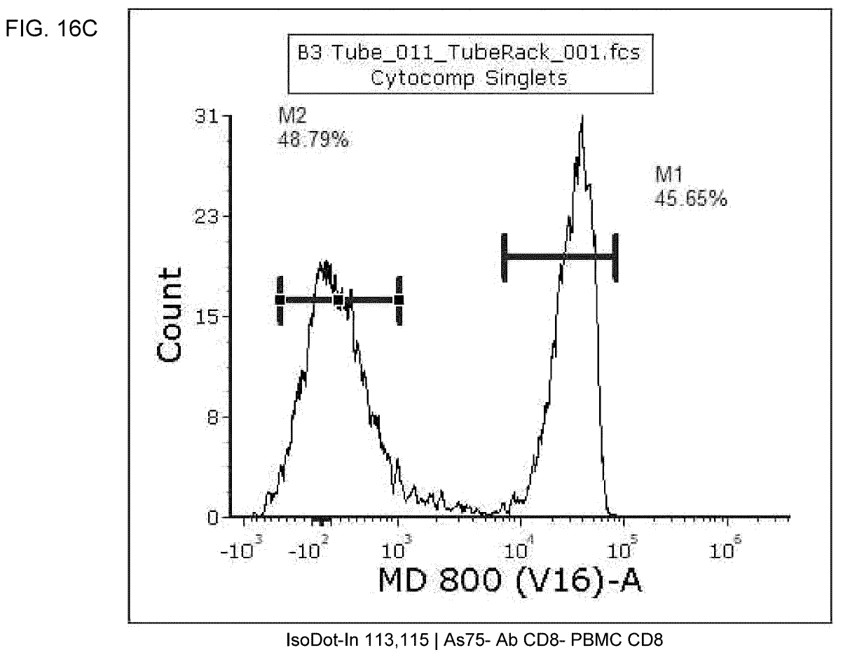

Now, with primary reference to FIG. 16A, the univariant histogram (florescence intensity versus particle number) depicts detection of CytoComp cells with CD45 receptors labelled with IsoDot-Au197-Abmouse anti-human CD45 followed by secondary label with Goat Anti-Mouse Alexa 488. The histogram evidences that 94.19% of the cells in the sample can be identified with IsoDot-Au197 Alexa Fluor 488-Ab mouse anti-human CD45-PBMC CD45.

Now, with primary reference to FIG. 16B, the univariant histogram (florescence intensity versus particle number) depicts detection of CytoComp cells with CD8 receptors labelled with IsoDot-CdSe-Ab mouse anti-human CD8 and analyzed with a Cytek® Aurora Spectral Flow Cytometer. The histogram evidences that 10.57% of the cells in the sample can be identified with IsoDot-CdSe-Ab mouse anti-human CD8.

Now, with primary reference to FIG. 16C, the univariant histogram (florescence intensity versus particle number) depicts detection of Cytocomp cells with CD4 receptors labelled with InAs 840 mouse anti-human CD4 IsoDot and analyzed in Cytek® Aurora Spectral Flow Cytometer. The histogram evidences that 45.65% of the cells in the sample can be identified with InAs 840 mouse anti-human CD8 IsoDot

### Cell Labelling Utilizing MagDots-Ab.

MagDots-Ab (6b) can also effectively and specifically label cellular targets (24′). The MagDots-Ab (6b) can effectively and specifically label cell surface receptors and subcellular structures in both live or fixed cells without any detectable non-specific binding. As one illustrative example of MagDots-Ab (6b) labeling of cellular targets (24′), peripheral blood mononuclear cells (“PBMC”) (24′) can be labeled using MagDots-Ab (6b). As one example, a scalable method can include one or more of: diluting peripheral blood as an iso-osmatic solution 50:50 with Hank's balanced salt solution (“HBSS”) (SigmaAldrich, PN H6648). Diluted peripheral blood can be layered over a Ficoll-Hypaque gradient (density=1.077 g/cm³) and centrifuged at 1350 RPM for 30 min. without brake. Serum can be aspirated and discarded. PBMC can be removed and transferred to a PBMC collection tube and washed with PBS. The PBMC pellet can be resuspended in 10 mL PBS and cell counts performed on a hemacytometer. A million PBMC (7) in 200 μl of PBS can be introduced into a 12×75 mm cytometry tube to which 20 μL of MagDots-mouse anti human CD3 (6b) were added. PBMC and MagDots-Ab mouse anti human CD3 (6b) are incubated for 25 min. at RT. After expiration of the incubation period, 200 μl of additional PBS can be added to the MagDots-Ab mouse anti human CD3 bound PBMC (7) (“MagDots-Ab mouse anti human CD3-PBMC”) in the cytometry tube.

### Magnetic Separation Utilizing MagDots-Ab

Place the flow cytometry tube containing MagDots-Ab mouse anti human CD3-PBMC (7) in PBS in a magnetic field gradient (23) (e.g. up to about 100-200 T/m). After expiration of 15 min., the nonmagnetic fraction can be aspirated from the flow cytometry tube and placed a nonmagnetic fraction collection tube. The flow cytometry tube containing the magnetically retained MagDots-mouse anti human CD3-PBMC (7) can be removed from the magnetic field gradient and the MagDots-mouse anti human CD3-PBMC (7) can be resuspended in 400 μl PBS. The PBMC/MagDots-mouse anti human CD3 reaction solution prior to magnetic separation, the nonmagnetic fraction after magnetic separation, and the MagDots-Ab mouse anti human CD3-PBMC (7) fraction after magnetic separation, were each analyzed by flow cytometer (8).

### Flow Cytometry Utilizing MagDots-Ab

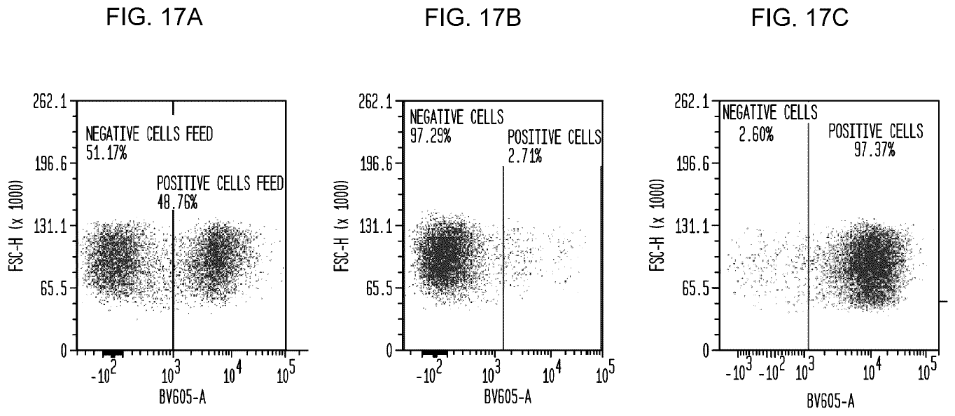

Now, referring primarily to FIGS. 17A through 17C, which depict bivariant dot plots (forward scatter area versus forward scatter height) obtained by flow cytometry analysis (8) of each of the PBMC/MagDots Ab-mouse anti human CD3-PBMC reaction solution prior to magnetic separation (as shown by the example of FIG. 11A), the non-magnetic fraction after magnetic separation (as shown by the example of FIG. 11B), and the MagDots-Ab mouse anti human CD3-PBMC fraction after magnetic separation (as shown by the example of FIG. 11C). The flow cytometry dot plots evidence the proportion of CD3 negative cells to CD3 positive cells. As evidenced by FIG. 11A, the PBMC/MagDots-mouse anti human CD3-PBMC reaction solution prior to magnetic separation contains 51.17% CD3 negative cells and 48.76% CD3 positive cells. FIGS. 11B and 11C, evidence that after magnetic separation the non-magnetic fraction contains largely CD3 negative cells 97.29% and very few CD3 positive cells 2.71%, whereas by contrast, FIG. 11C evidence that the MagDots-Ab mouse anti human CD3-PBMC fraction after magnetic separation includes very few CD3 negative cells 2.60% and largely CD3 positive cells 97.37%. This evidences the substantial advantage of using MagDots-Ab (6b) to capture and purify cellular targets (24′).

### Mass Cytometry Utilizing IsoDots-Ab-PBMC

Mass cytometry is reviewed in Tanner et al., Cancer Immunol Immunother (2013) 62:955-965 and Spitzer and Nolan, Cell (2016) 165 (4): 780-91, both of which are hereby incorporated by reference herein in their entirety.

Analysis of IsoDots-Ab mouse anti human CD8-PBMC (7) prepared as above described were analyzed by mass cytometry. A mass cytometer suitable for use in the analysis of IsoDots-Ab mouse anti human CD8-PBMC (7) can comprise a Helios CyTOF® system manufactured by Standard Biotools, 2 Tower Place, Suite 2000, South San Francisco, CA 94080. The analysis of the data acquired can be analyzed using appropriate software. A software suitable for the analysis of data acquired by the mass cytometer is FlowJo software distributed by FlowJo, LLC, 385 Williamson Way, Ashland, Oregon 97520. The operation of a mass cytometer is in accordance with the instructions provided in the manufacturer's user guide.

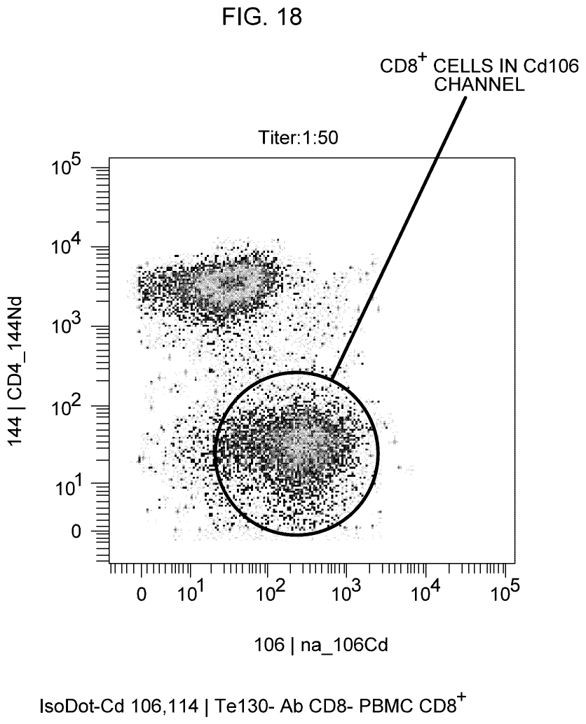

Now, with primary reference to FIGS. 18 through 20, which provide illustrative examples of utilizing time-of-flight mass cytometry to analyze IsoDots-Ab mouse anti human CD8-PBMC (7) tagged with metal isotopes cadmium-106, cadmium-114 and tellurium-130. IsoDots-Ab mouse anti human CD8-PBMC (7) tagged with metal isotopes cadmium-106, cadmium-114 and tellurium-130 were nebulized, and then vaporized and atomized. The resulting ion cloud was passed through a quadrupole to discard common biological elements, enriching for the metal ion reporters cadmium-106, cadmium-114 and tellurium-130. Cadmium-106, cadmium-114 and tellurium-130 were then separated based on their mass-to-charge ratio in the time-of-flight mass spectrometer. Counts of metal ions from metal ion clouds from single cells were converted to electrical signals which were then analyzed as a data matrix for single-cell characterization of multiple cellular parameters. The software translated these digital signals into interpretable data points that indicated differentiation of different metal isotopes cadmium-106, cadmium-114 and tellurium-130 to the mouse anti human CD8 antibodies. The software further converted the data points in visual lower dimensional space as dot plots, with each dot representing a single CD8 cell event. Each axis displays the signal intensity for a different metal isotope reporter. Cells with similar expression levels cluster together, allowing cell populations to be differentiated based on their unique marker profiles. In mass cytometry “channels” refer to the distinct, mass-resolved signals generated by the metal isotope-conjugated antibodies. Each channel corresponds to a unique atomic mass detected by the mass cytometer, allowing for the simultaneous measurement of different metal ion reporters associated with single cells without the spectral overlap.

Now, with primary reference to FIG. 18 which includes a dot plot of IsoDots-Ab mouse anti human CD8-PBMC (7) cell events in the cadmium-106 channel. The CD8 positive cell events are represented by the encircled cluster labeled CD8+ cells Cd-106 Channel. This CD8+ Cd-106 cluster is evidence that the IsoDot-mouse anti human CD8-PBMC can be successfully prepared as above described and utilized in mass cytometry to differentiate cell populations based on the signal intensity of the metal isotope reporter, in this instance Cd-106.

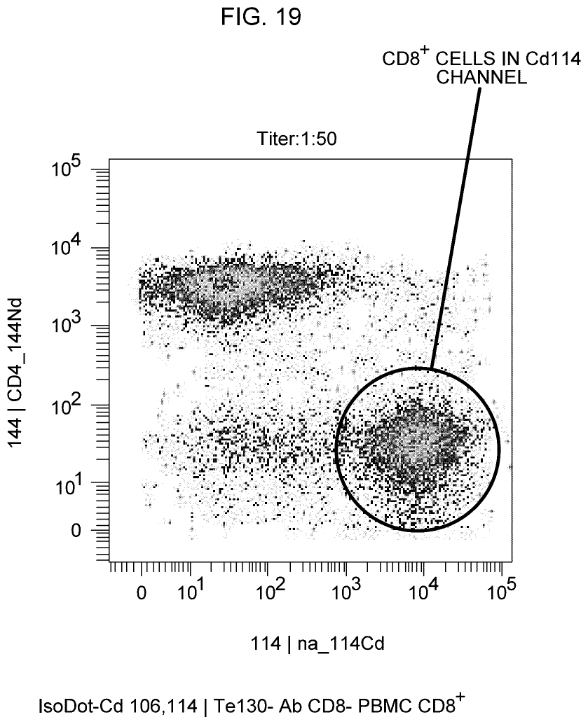

Now, with primary reference to FIG. 19 which includes a dot plot of IsoDots-Ab mouse anti human CD8-PBMC (7) cell events in the cadmium-114 channel. The CD8 positive cell events are represented by the encircled cluster labeled CD8+ cells Cd-114 Channel. This CD8+ cluster is evidence that the IsoDot-mouse anti human CD8-PBMC can be successfully prepared as above described and utilized in mass cytometry to differentiate cell populations based on the signal intensity of the metal isotope reporter, in this instance Cd-114.

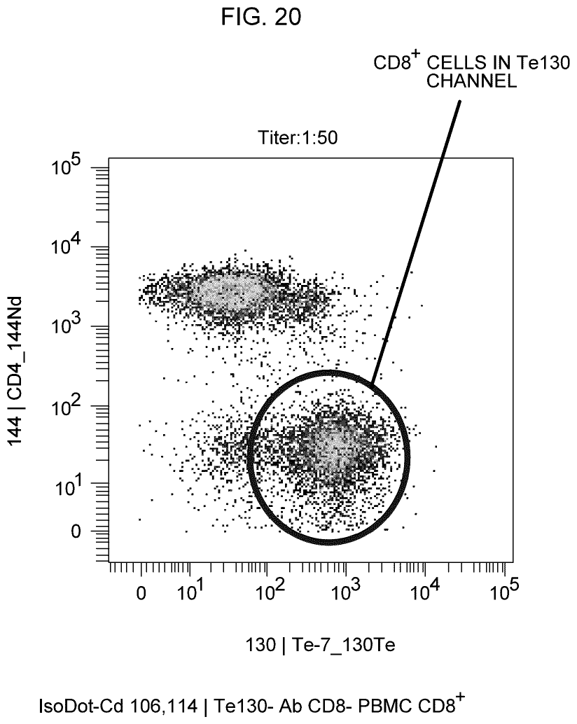

Now, with primary reference to FIG. 20 which includes a dot plot of IsoDots-Ab mouse anti human CD8-PBMC (7) cell events in the tellurium-130 channel. The CD8 positive cell events are represented by the encircled cluster labeled CD8+ cells Te-130 Channel. This CD8+ cluster is evidence that the IsoDot-mouse anti human CD8-PBMC can be successfully prepared as above described and utilized in mass cytometry to differentiate cell populations based on the signal intensity of the metal isotope reporter, in this instance Te-130.

Again, with primary reference to FIGS. 18 through 20, the dot plot of IsoDots-mouse anti human CD8-PBMC cell events concurrently in the cadmium-106 channel, the cadmium-114 channel, and the tellurium-130 channel is evidence that IsoDots prepared as above described can encapsulate one or concurrently a plurality of different metal isotopes (as shown in the examples of FIGS. 7 through 10). Moreover, IsoDots have a stereoscopic volume that efficiently diffuses through cell membranes and can be loaded with metal isotope atoms of one or concurrently a plurality of different metal isotopes to afford, when conjugated to an antibody and subsequently bound to a cell, signal intensity in each mass cytometry channel sufficient to measure even weakly expressed markers.

As can be easily understood from the foregoing, the basic concepts of the present invention may be embodied in a variety of ways. The invention involves numerous and varied embodiments of polymer encapsulated nanoparticles and methods for making and using such polymer encapsulated nanoparticles.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of an “encapsulated nanoparticle” should be understood to encompass disclosure of the act of “encapsulating a nanoparticle”—whether explicitly discussed or not—and, conversely, were there effectively disclosure of the act of “encapsulating a nanoparticle”, such a disclosure should be understood to encompass disclosure of a “encapsulated nanoparticle” and even a “means for encapsulating a nanoparticle.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

Thus, the applicant(s) should be understood to claim at least: i) each of the polymer encapsulated nanoparticles herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application, if any, provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon. The elements following an open transitional phrase such as “comprising” may in the alternative be claimed with a closed transitional phrase such as “consisting essentially of” or “consisting of” whether or not explicitly indicated the description portion of the specification.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A nanocomposite, comprising:
an amphiphile having a hydrophobic region and a hydrophilic region, said amphiphile comprises polystyrene-b-polyethylene glycol including a functional group,
wherein said polystyrene-b-polyethylene glycol includes a polystyrene block and a polyethylene glycol block including a branched polyethylene glycol selected from the group of: a four arm polyethylene glycol, a six arm polyethylene glycol and an eight arm polyethylene glycol; and
at least one inorganic particle encapsulated by association with said hydrophobic region of said amphiphile.

2. The nanocomposite of claim 1, wherein said at least one inorganic particle comprises one or more of: metal-based particles including a metal oxide particle, a metal halide particle, a doped metal, a metal isotope particle, a non-metal isotope particle, a quantum dot, a Perovskite quantum dot, and combinations thereof.

3. The nanocomposite of claim 1, wherein said at least one inorganic particle comprises a plurality of metal isotope particles having the same atomic mass.

4. The nanocomposite of claim 1, wherein said at least one inorganic particle comprises a plurality of metal isotope particles having different atomic mass.

5. The nanocomposite of claim 1, wherein said at least one inorganic particle comprises a plurality of metal particles having the same number of protons but a different number of neutrons.

6. The nanocomposite of claim 1, wherein said at least one inorganic particle comprises at least one isotope particle selected from the group consisting of:
As-75
Se-74, Se-76, Se-77, Se-78, Se-80, Se-82
Br-79, Br-81
Y-89
Zr-90, Zr-91, Zr-92, Zr-94, Zr-96
Mo-92, Mo-94, Mo-95, Mo-96, Mo-97, Mo-98, Mo-100
RE-96, RE-98, RE-99, RE-100, RE-101, RE-102, RE-104
Rh-103
Pd-102, Pd-104, Pd-105, Pd-106, Pd-108, Pd-110
Ag-107, Ag-109; Cd-48
Cd-106, Cd-108, Cd-101, Cd-111, Cd-112, Cd-113, Cd-114, Cd-116;
In-113, In-115
Sn-112, Sn-114, Sn-115, Sn-116, Sn-117, Sn-118, Sn-119, Sn-120, Sn-122, Sn-124
Sb-121, Sb-123
Te-120, Te-122, Te-123, Te-124, Te-125, Te-126, Te-128, Te-130
Iodine: I-127
Cs-133
Ba-130, Ba-132, Ba-134, Ba-135, Ba-136, Ba-137, Ba-138
La-138, La-139
Ce-136, Ce-133, Ce-140, Ce-142
Pr-141
Nd-142, Nd-143, Nd-144, Nd-145, Nd-146, Nd-148, Nd-150
Sm-144, Sm-147, Sm-148, Sm-149, Sm-150, Sm-152, Sm-154
Eu-151, Eu153
Gd-152, Gd-154, Gd-155, Gd-156, Gd-157, Gd-158, Gd-160
Tb-159
Dy-156, Dy-158, Dy-160, Dy-161, Dy-162, Dy-163, Dy-164
Ho-165
Er-162, Er-164, Er-166, Er-167, Er-168, Er-170
Tm-169
Yb-168, Yb-170, Yb-171, Yb-172, Yb-173, Yb-174, Yb-176
Lu-175, Lu-176
Hf-174, Hf-176, Hf-177, Hf-178, Hf-179, Hf-180
Ta-180, Ta-181
W-180, W-182, W-183, W-184, W-186
Re-185, Re-187
Pt-190, Pt-192, Pt-194, Pt-195, Pt-196, Pt-198
Au-197
Hg-196, Hg-198, Hg-199, Hg-200, Hg-201, Hg-202, Hg-204
Pb-204, Pb-206, Pb-207, Pb-208
Bi-209.

7. The nanocomposite of claim 1, further comprising at least one magnetic particle encapsulated by association with said hydrophobic region of said amphiphile.

8-11. (canceled)

12. The nanocomposite of claim 1, further comprising at least on quantum dot encapsulated by association with said hydrophobic region of said amphiphile.

13-18. (canceled)

19. The nanocomposite of claim 1, further comprising a linker bound to said functional group of said nanocomposite.

20. The nanocomposite of claim 19, wherein said linker comprises a heterobifunctional linker having a first reactive group adapted to react with said functional group of said amphiphile encapsulating said nanoparticle and having a second reactive group adapted to react with an agent.

21. (canceled)

22. The nanocomposite of claim 20, wherein said functional group comprises an amine and said first reactive group of said heterobifunctional linker comprises a succinimidyl carboxymethyl ester.

23. (canceled)

24. (canceled)

25. The nanocomposite of claim 20, further comprising an agent that binds to said second reactive group, wherein said agent is selected from the group consisting of: polyethylene glycol, an antibody, a half antibody, an antibody fragment, a fluorescent probe, an aptamer, a vitamin, a cell surface receptor, a cell coat, a protein, a peptide, a radioactive isotope, a contrast media, a surface charge modifier, a lectin, and combinations thereof.

26. The nanocomposite of claim 19, further comprising an antibody or an antibody fragment bound to said linker bound to said functional group of said nanocomposite.

27. The nanocomposite of claim 26, wherein said antibody or an antibody fragment bound to said linker bound to said functional group of said nanocomposite adapted to bind a cellular target.

28. (canceled)

29. The nanocomposite of claim 27, wherein said antibody or said antibody fragment bound to said linker bound to said functional group of said nanocomposite adapted to bind said cellular target without detectable non-specific binding of other molecules.

30. The nanocomposite of claim 27, wherein said nanocomposite linked to said antibody or antibody fragment bound to said cellular target is analyzed by flow cytometry for detection of said cellular target.

31. The nanocomposite of claim 7, further comprising a magnetic field wherein said nanocomposite linked to an antibody or an antibody fragment binding said cellular target is placed in a magnetic field to isolate said nanocomposite linked to said antibody or antibody fragment binding said cellular target.

32. The nanocomposite of claim 31, wherein said nanocomposite linked to said antibody or antibody fragment binding said cellular target isolated by placement in said magnetic field analyzed by flow cytometry for detection of said cellular target.

33. The nanocomposite of claim 26, wherein said antibody or antibody fragment comprises mouse anti human CD and a cellular target comprises a human CD peripheral blood mononuclear cells.

34-46. (canceled)

47. The nanocomposite of claim 1, further comprising an agent that binds to said functional group, said agent adapted to bind a cellular target.

48. The nanocomposite of claim 47, wherein said agent bound to said cellular target analyzed by mass cytometry.

49. The nanocomposite of claim 1, further comprising:
a linker bound to said functional group of said nanocomposite; and
an agent bound to said linker, said agent adapted to bind a cellular target.

50. The nanocomposite of claim 49, wherein said agent bound to said cellular target analyzed by mass cytometry.

51. The nanocomposite of claim 48, wherein said agent bound to said cellular target comprises an antibody or an antibody fragment.

52. The nanocomposite of claim 51, wherein said antibody or an antibody fragment bound to said cellular target analyzed by mass cytometry.

53. The nanocomposite of claim 52, wherein said cellular target comprises a CD cell.

54. The nanocomposite of claim 53, wherein said CD cell is selected from the group consisting of: a CD3 cell, a CD4 cell, a CD8 cell, a CD16 cell and a CD32 cell.
